(19) United States
(12) Patent Application Publication
(10) Pub. No.: US 2012/0040366 A1
(43) Pub. Date: Feb. 16, 2012

(54) COMPOSITIONS, METHODS AND USES FOR DISEASE DIAGNOSIS

(75) Inventors: Jeffrey Holt, Harvey's Lake, PA (US); Kimberly A. Gibson, Denver, CO (US)

(73) Assignee: Tissue Genetics, Inc., Aurora, CO (US)

(21) Appl. No.: 13/148,931

(22) PCT Filed: Feb. 10, 2010
(86) PCT No.: PCT/US10/23800

§ 371 Date: Nov. 7, 2011

Related U.S. Application Data
(60) Provisional application No. 61/151,337, filed on Feb. 10, 2009.

(51) Int. Cl. C12Q 1/68; G01N 33/566; C12P 19/34; C12N 1/21; C07H 21/02; C07K 14/435; C07K 7/08; C07K 16/18; C12N 15/12; G01N 33/574; C12N 15/63

(52) U.S. Cl. 435/6.14; 435/7.23; 435/7.92; 435/91.1; 435/252.33; 435/320.1; 530/324; 530/326; 530/327; 530/350; 530/387.3; 530/388.2; 530/389.1; 530/391.1; 536/23.5; 536/24.5

(57) ABSTRACT

Embodiments of the present invention generally related to methods and compositions for diagnosing or predicting a genetic disorder. In certain embodiments, the methods may include use of rapid and inexpensive assay systems. Other embodiments concern novel mutation specific peptides associated with BRCA1 or BRCA2. In yet other embodiments, haploid cells are used to diagnose a genetic disorder in a subject.

### CROSS-REFERENCE TO RELATED APPLICATIONS

This application claims the benefit of U.S. Provisional Patent Application Ser. No. 61/151,337, filed on Feb. 10, 2009, which is incorporated by reference herein for all purposes.

### FIELD

Embodiments of the present inventions generally relate to compositions, methods and uses for assessing presence or risk of developing a genetic disorder in a subject. In some embodiments, methods and compositions are reported for diagnosing or predicting the onset of a genetic disorder and/or therapeutic treatment for and/or progression of the disorder. In other embodiments, methods for identifying, predicting the onset of a genetic disorder, or preventing the development of a genetic disorder based en detection or levels of novel proteins are disclosed. Certain embodiments of the present invention concern screening haploid cells for assessing predisposition or onset of a disorder.

### BACKGROUND

An increasing number of genes that play a role in many different disorders are being identified. In certain disorders, detection of mutations in these genes is instrumental in determining susceptibility to or diagnosing the presence of a disorder.

Inherited mutations which are responsible for breast cancer include both common founder mutations and a great number of diverse less common mutations. For example, 3% of Ashkenazi Jews have one of two common founder mutations which are responsible for as much as 50% of ovarian cancer in Israel. In many other cases individuals affected by a given disease display extensive allelic heterogeneity. For example, more than 125 mutations in the human BRCA1 gene have been reported. Mutations in the BRCA1 gene are thought to account for roughly 45% of inherited breast cancer and 80-90% of families with increased risk of early onset breast and ovarian cancer.

Breast cancer is also an example of a disease which has genetic heterogeneity. In addition to BRCA1, the BRCA2 and BRCA3 genes have been linked to breast cancer. Similarly, the NFI and NFII genes are involved in neurofibromatosis (types I and II, respectively).

Several other markers specific for particular tumor or cancer types have been used with increasing frequency over the last five years. BRCA1 and BRCA2 in the context of breast and ovarian cancer are examples of such markers.

Despite many scientific advances in recent years to better predict disorders on the level of gene mutations, such disorders continue to cause long-term disability in a significant number of patients. The ability to predict the potential onset or predisposition to a disorder is an important goal for patients and their treating clinicians in order to maximize the potential for early intervention and monitoring of the patient.

### SUMMARY

Some embodiments of the present invention relate to methods compositions and uses for detection of, or predicting predisposition for, a genetic disorder in a subject. In certain embodiments, novel peptides and novel proteins have been identified of use in diagnosing, predicting or treatment of certain genetic disorders. In certain embodiments, amino acid sequences are disclosed including, but not limited to SEQ ID NOs: 1, 2, 3, 4, 5, 6, 7, 8, 9 and 10. In accordance with these embodiments all or part of the novel regions of these sequences can be used in any embodiments disclosed herein. For example, one or more tissue, tumor, or germ cell samples may be obtained from a subject and one or more of the samples can be assessed with respect to one or more target protein(s), peptides or nucleic acid molecules. For example, one or more sample may be analyzed for the presence or level of one or more target protein(s) by any assay known in the art. In certain embodiments, one or more antibodies, or one or more aptamer(s) or one or more nucleic acid sequence(s) capable or known to associate with novel peptides disclosed herein may be used in an assay. Antibodies may include, but not limited to, mutation specific antibodies, carboxy-terminal antibodies, amino-terminal antibodies, or antibody fragments selected to bind to proteins or peptides disclosed herein.

Other embodiments concern generating constructs for expression of compositions disclosed herein. For example, constructs may be used to generate a transgenic cell that expresses the disclosed proteins or peptides.

In certain embodiments, detection of a genetic disorder in a subject may include detection of propensity to a recessive genetic disorder or a dominantly inherited genetic disorder in a subject not previously diagnosed with the disorder. In accordance with these embodiments, one or more samples may be obtained from a subject for analysis. These samples may include, but are not limited to breast, prostate, ovarian, pancreatic, lung brain, thyroid, bowel, skin and throat samples from a subject suspecting of having or developing a genetic disorder. These samples may be used to assess the level, state or alteration of one or more target proteins or peptides present in the tissue sample. In certain embodiments, these samples will be compared to control samples.

In some embodiments, methods, compositions and uses may include analysis of BRCA1 and/or BRCA2 in a subject suspected of having or developing cancer. In certain embodiments, BRCA1 or BRCA2 founder mutations may be analyzed. Some embodiments concern isolated proteins and/peptides of use for diagnosing or prognosing breast cancer in a subject. In accordance with these embodiments, certain hereditary breast cancers can have a one or two nucleotide deletion in a gene (e.g. BRCA1 or BRCA2 gene). A target protein of a sample can be targeted with a mutation-specific antibody capable of binding a mutation specific target protein or peptide region, for example, in order to assess level or presence of the mutation-specific target protein. In one embodiment, a deletion leads to a frame shifting of a triplet codon which can lead to generation of a completely different amino acid sequence and a novel protein.

In certain embodiments, composition and methods report agents that bind to novel Ashkenazi BRCA1 mutation 185delAG protein, peptide and corresponding nucleic acid sequences or fragments thereof. Other embodiments concern agent composition and methods for analyzing novel bind to novel Askenazi BRCA2 mutation 6174delT protein, peptide and corresponding nucleic acid sequences or fragments thereof. In other embodiments, compositions and methods concern agents that bind to novel proteins generated by founder mutations (e.g. BRCA1 and BRCA2).

In some embodiments, a target protein of a germ cell sample (sperm or oocytes) may be targeted with a carboxy-terminal directed antibody and an amino-terminal directed antibody to assess the ratio of carboxy-terminal directed antibody binding levels to amino-terminal directed antibody binding levels-to determine if a truncated protein is present in the germ cell. In accordance with these embodiments, the level of binding of the antibody or the ratio of binding between the different antibodies may be used to assess the risk of developing a disorder or detecting the presence of a previously undiagnosed disorder in the subject.

Other embodiments report analyzing a subject having a genetic disorder and assessing response to a treatment regimen for the genetic disorder by analyzing one or more sample(s) from the subject for presence or levels of one or more novel peptides or proteins or corresponding nucleic acid treatment thereof, created by founder mutations in a particular gene linked to the genetic disorder. In accordance with these embodiments, the regimen can be modulated depending on the novel peptide or protein levels. In certain embodiments, a mutation specific antibody as used herein may be directed to bind one or more of novel peptides of the novel target protein region produced as a consequence of the mutation (eg frameshift, deletion, insertion).

In one embodiment, examples of a mutation specific antibody may include a composition of the present invention, 185delAG mutation specific antibody. In accordance with this example, the level of binding of the 185delAG mutation specific antibody may be analyzed and the bound antibody may be correlated with potential responsiveness to a therapeutic agent such as a chemotherapeutic agent. Examples of therapeutic treatments include but are not limited to targeting BRCA1 or BRCA2 function using a therapeutic agent, using homologous recombination therapy, using radiation therapy, platinum-base drugs, or using drugs that inhibit DNA repair. Examples of therapeutic agents include, but are not limited to, homologous recombination therapy examples: mitomycin C, cis-platinum, carboplatinum, PARP inhibitors including 3-amino-benzamide, 8-hydroxy-2-methylquinazolin-4-(3H)-one (NU1025), AG14361; radiation therapy examples: including direct beam radiation, implanted source radiation, focused or refined beam radiation; agent inhibiting DNA repair including doxorubicine, cyclophosphamide, actinomycin D, bleomycin, irinotecan, and cis-platinum.

Other embodiments may include kits for detecting peptides or proteins contemplated herein. For example, kits may include one or more detection molecule for detecting all or a portion of SEQ ID NOs: 1, 2, 3, 4, 5, 6, 7, 8, 9 or 10. In accordance with these embodiments, a kit may be used to detect presence, propensity or progression of cancer in a subject.

### BRIEF DESCRIPTION OF THE DRAWINGS

The following drawings form part of the present specification and are included to further demonstrate certain embodiments of the present invention. The embodiments may be better understood by reference to one or more of these drawings in combination with the detailed description of specific embodiments presented herein.

FIG. 1 represents an exemplary flow chart demonstrating novel amino acids present in the novel polypeptides resulting from the common Ashkenazi founder mutations of BRCA1 and BRCA2.

FIG. 2 represents an exemplary schematic of hereditary and sporadic cancer-related protein modifications.

FIGS. 3A-3B represent antibodies bound to exemplary founder mutation peptides or proteins and control samples.

### DESCRIPTION OF ILLUSTRATIVE EMBODIMENTS

### Definitions

As used herein, “a” or “an” may mean one or more than one of an item.

As used herein, “modulation” refers to a change in the level or magnitude of an activity or process. The change may be either an increase or a decrease. For example, modulation may refer to either an increase or a decrease in activity or levels. Modulation may be assayed by determining any parameter that indirectly or directly affects or reflects truncation of a protein or a change in post-translational modification of a protein.

As used herein, “founder mutation” can mean a mutation that appears in the DNA of one or more individuals who are founders of a distinct population. Founder mutations can initiate with changes that occur in the DNA and can get passed down to other generations.

### DETAILED DESCRIPTION

In the following section, various exemplary compositions and methods are described in order to detail various embodiments of the invention. It will be obvious to one skilled in the art that practicing the various embodiments does not require the employment of all or even some of the specific details outlined herein, but rather that concentrations, times and other specific details may be modified through routine experimentation. In some cases, well known methods or components have not been included in the description.

Embodiments of the present invention relate to compositions, methods and uses for early detection of predisposition for, or prevention of a genetic disorder in a subject.

Healthcare providers are in need of an inexpensive, rapid and easy method to detect genetic alteration(s) of target proteins known to confer predisposition to a genetic disorder. Because of the nature of genetic disorders, such as cancer, as a dynamic on-going process, a method that can rapidly track a mutational change would be extremely beneficial from a clinical perspective. The application of such methods is important for subject having or with a propensity for a recessive genetic disorder or a dominantly inherited genetic disease like cancer.

In certain embodiments, founder mutations were analyzed for potential read-through peptides instead of truncations, as commonly considered. Those skilled in the art have focused on truncation of for example, BRCA1 and BRCA2, whereas embodiments disclosed herein focus on read-through amino acids as a result of a mutation. In certain examples, studies disclosed herein regarding mutations of BRCA1 and BRCA2 found in certain populations lead to the discovery of novel peptide regions and novel proteins generated from mutations contemplated in these genes. These peptides and proteins are disclosed. Antibodies and other detection molecules were generated to the novel regions for further study, analysis and assay development (see EXAMPLES).

In some embodiments of the present invention, a health care provider can obtain one or more samples(s) from a subject having or with a propensity for a recessive genetic disorder or a dominantly inherited genetic disease. Samples contemplated herein may include but are not limited to breast, prostate, ovarian, pancreatic, lung brain, thyroid, bowel, skin, gastrointestinal, buccal, germ cell, and throat tissue samples. Examples of genes associated with disorders consider herein include, but are not limited to BRCA2, BRCA1, Familial Polyposis (FAP), Duchenne's Muscular Dystrophy, Beta-thalassemia.

In certain embodiments, a BRCA1 mutation lead to discovering a novel 16 amino acid sequence which is not found in BRCA1 polypeptide and a search of computer databases demonstrated that it does not occur in nature except for the case of this mutation. In other embodiments, a common Askenazi BRCA2 mutation 6174delT lead to discovering a novel 21 amino acid sequence which is not homologous to the BRCA2 polypeptide and a search of computer databases demonstrated that it does not occur in nature. Thus, these are not altered BRCA1/BRCA2 polypeptides but novel polypeptides produced specifically by the founder mutation for which analytical detection molecules have been generated. In some embodiments, about 50% of the amino acid sequences are contemplated for use in an assay, in other embodiments about 60%, or about 70%, or about 80% or about 90% or more is contemplated of use for an assay of a subject having, suspected of developing or with increased progression of a genetic disorder.

Some embodiments concern inherited mutations that can be responsible for genetic disorders or contribute to a genetic disorder in a subject having the mutation. For example, inherited mutations may lead to protein truncation or to proteins with altered amino acids or post translational modifications. Certain genetic diseases such as inherited breast or colon cancer are often a consequence of mutations which change an amino acid to a termination codon thus resulting in protein truncation. Methods to identify subjects or families with inherited disease has previously involved expensive and cumbersome methods: either complete gene sequences or PCR-based protein truncation tests which require a subject's blood samples and complicated and expensive technologies. These approaches also require a high index of suspicion that the disease exists and often few patients are identified since genetic susceptibility is often clinically silent.

Although immunohistochemistry is routinely employed for identifying biomarkers within tissue samples, compositions and methods herein include novel approaches to identify specific truncated proteins or peptides associated with mutations disclosed herein by screening for novel amino acid sequence or protein in a sample. In accordance with these embodiments, a novel protein or peptide can be created by a deletion which leads to a frameshift in the amino acid coding sequencing. In one embodiment, a compositions and methods disclosed herein can include, but are not limited to immunohistochemical methods which can be employed, for example in a hospital setting or forward to a laboratory for analysis. In addition, all subjects can be analyzed using more cost effective methods disclosed herein whereas it was previously required that a high index of suspicion be present before pursuing complicated methodologies. In addition, genetic disorders identified by compositions and methods disclosed herein have specific molecular causes and targeted therapies can be identified for subjects having these conditions. In general, genetic screening can be performed on patients without disease who have a strong familial history and wonder if they will likely develop the disease. Compositions and methods disclosed herein disclose simple and rapid screening processes applicable to anyone with or suspected of developing a disorder (e.g. sporadic or genetic cancer). In other embodiments, screening methods can be facilitated by knowing the specific ethnicity of the subject being screened. For example, founder mutations are by their very nature found in specific ethnic or geographic populations. In addition, compositions and methods disclosed herein can be used to identify those patients who have a genetic causation and can be used for family screening and carrier identification or therapeutic development to reduce the levels of causative genes or prevent onset of a genetic disorder.

In some embodiments, compositions and methods disclosed herein may be used to identify carriers of specific genetic diseases which allows targeted therapies, family screening, and prevention strategies to be identified for family members. In other embodiments, neutralizing targeting molecules may be used to target expression of nucleic acids or protein expression of a causative novel protein disclosed herein in order to reduce or prevent onset of a genetic disorder associated with the novel protein. Compositions and methods disclosed herein can reduce current labor intensive and costly testing techniques or serve as an additional test fbr potential false negative or false positive results in a dual assay system. In addition, truncations are difficult to identify and quantitate, therefore identify a definitive region of a protein (e.g. by presence or absence) can further clarify a patient's realtime status with respect to a genetic disorder. One advantage disclosed herein provides a simple sample-based test to identify patients with genetic mutations. Studies have shown that over 200,000 cases of breast cancer and 106,000 cases of colon cancer occur in the US each year. These are just two examples of the disorders that might benefit from the disclosed screening methods.

### Haploid Cell Assay

In other embodiments, compositions and methods were developed to identify for example, analyze sperm or eggs (oocytes) for certain DNA mutation which can lead to truncated proteins, then one could select for offspring that will not develop the specific inherited disease. Truncated proteins can be difficult to find in diploid cells because both truncated and wildtype proteins are present (because only one mutant chromosome is there). However, because germ cells including for example, sperm and oocytes are haploid and contain only one copy of each chromosome, the truncated protein is either present as the only protein form or not present at all. This facilitates screening for truncated proteins in germ cells and allows the development of methods to sort affected germ cells containing truncated proteins from those germ cells which do not contain truncated proteins. These embodiments can assist in an urgent need for new early detection, rapid and inexpensive approaches to diagnosis of recessive genetic or dominantly inherited genetic disorders, including but not limited to breast cancer, ovarian cancer, prostatic cancer, melanoma, skin cancer, blood disorders.

In some embodiments, a rapid, simple and inexpensive method can be used to identify samples which have a specific genetic mutation leading to protein truncation. This approach facilitates a simple and rapid identification of disease mutations in a subject not previously diagnosed with a disorder nor previously suspected of having the disorder. It is contemplated herein that any assay methods known the art may used to assay haploid cells for truncated or mutant protein or peptide. Some examples other combination technologies may include, but are not limited to, DNA sequencing, microarray technologies, tissue biopsies, ultrasound technologies, patient history, presence of co-existing diseases, IHC for other proteins or markers, histopathologic staging, or other methods, These additional methods may be used, for example, to confirm a diagnosis, assess effectiveness or development of a therapy or predict the risk of a disorder in an asymptomatic subject.

Some embodiments of the present invention relate to methods to analyze a tissue sample in order to detect truncation of a target protein. These methods can include a simple, inexpensive and rapid analysis of tissue samples. In accordance with these embodiments, a tissue sample can be fresh, frozen, a homogenate or fixed archival. In addition, this simple and rapid analysis may be used to diagnosis recessive genetic disorders or a dominantly inherited genetic disorders. Examples of these disorders include but are not limited to blood diseases, muscular dystrophy and cancers such as breast and ovarian cancer.

Table 1. Exemplary Mutation Specific Peptide Regions from BRCA1 and BRCA2

### Mutation Specific Antibody Development

TABLE 1
Exemplary Mutation Specific Peptide Regions from
BRCA1 and BRCA2

| | Protein | |
|---|---|---|
| | Expressed: | |
| | Amino Acid | |
| BRCA allele | Sequence | Novel Sequence |
| BRCA1-185delAG | SEQ ID NO: 6 | VSHLSGVDQGTCLHKV |
| | | (SEQ ID NO: 1) |
| BRCA2-6174delT | SEQ ID NO: 7 | RENLSRYQMLHYKTQDKCFLK |
| | | (SEQ ID NO: 2) |
| BRCA1-943ins10 | SEQ ID NO: 8 | AMWHKYSCQLITA |
| | | (SEQ ID NO: 3) |
| BRCA1-5382insC | SEQ ID NO: 9 | PGQKDLQGARNLLLWALHQHA |
| | | HRSTGMDGTAVWCFCGEGAFI |
| | | IHPWHRCPPNCGCAARCLDRG |
| | | QWLPCNWADV |
| | | (SEQ ID NO: 4) |
| BRCA1-4153delA | SEQ ID NO: 10 | KEERAWKKIIKKSKAWIQT |
| | | (SEQ ID NO: 5) |

In previous studies, common founder mutations have been identified in a number of ethnic groups. These include Ashkenazi Jews, African-Americans, Hispanic Americans, Polish and Dutch patients. Monoclonal antibodies were developed which specifically recognized the novel amino acids resulting from the common Ashkenazi founder mutations 185delAG-BRCA1 and 6174delT-BRCA2. Data revealed that these antibodies to not react with non-mutant cells or tissues by immunohistochemistry. These antibodies are directed to bind to 6174delT-BRCA2 or 185delAG-BRCA1 novel peptides or proteins respectively. In one exemplary embodiment, formalin-fixed, paraffin embedded tissues from a 185delAG-BRCA1 mutant hereditary breast cancer patient were exposed to the antibody and demonstrated that the normal tissues and cancer expressed the mutant protein. These cancers would likely benefit from BRCA1/BRCA2 targeted therapies.

Some embodiments herein disclose novel proteins (e.g. SEQ ID NOs. 6-10) and novel peptide regions associated with BRCA1 or BRCA2 (e.g SEQ ID NOs. 1-5) of use in compositions and methods disclosed herein. In certain embodiments, novel amino acid sequences for 185delAG-BRCA1 (SEQ ID NO: 1) or 6174delT-BRCA2 (SEQ ID NO: 2) were synthesized.

In some embodiments herein, one or more mutation specific antibodies can be used to screen one or more sample(s) from a subject for the level or presence of novel amino acids of one or more target specific mutant proteins known to associate with a genetic disorder. In accordance with these embodiments, response of a subject to a predetermined therapeutic treatment for the disorder can be evaluated. In one embodiment, a mutation specific antibody can be used to screen a tissue sample of a subject for response to a therapy, for example PARP inhibitors including 3-amino-benzamide, 8-hydroxy-2-methylquinazolin-4-(3H)-one (NU1025), AG14361 or combination thereof or other treatment known in the art.

In one embodiment, level of binding of a mutation specific antibody to a tissue sample target protein can be assessed using a rapid screening technique. Examples of these techniques can include current technologies such as immunohistochemistry, western blot analysis, ELBA, immunoprecipitation, radioimmunoassay, mass spectroscopy, gas-chromatography-mass spectroscopy, two-dimensional electrophoresis and staining with organic dyes, metal chelates, fluorescent dyes, complexing with silver, or pre-labeling with fluorophores. In accordance with these embodiments, the level of mutation specific antibody may be used to distinguish genetic variations of a disorder such as cancer. A better understanding of the genetic variation of a disorder can lead to a more accurate diagnosis and prognosis as well as a more tailored therapeutic treatment for a subject having or suspected of developing a disorder.

In other embodiments, the techniques disclosed herein may be used as an initial screening process for having or risk of developing a disorder. In some embodiments, ethnic population may be screened for identification of a founder mutation novel peptide or protein disclosed herein. Upon completion of the screening for mutation specific antibody binding to a target protein and/or detection of truncation of a target protein, the levels of binding of the antibodies to a tissue sample may be used to evaluate whether further testing is necessary, immediate intervention is required or future evaluations and monitoring are required by a healthcare professional to evaluate the subject.

An “antibody” as used herein can refer to a full-length (e.g., naturally occurring or formed by normal immunoglobulin gene fragment recombinatorial processes) immunoglobulin molecule (e.g., an IgG antibody) or an immunologically active (e.g., specifically binding) portion of an immunoglobulin molecule, like an antibody fragment. The term “antibody” also includes “humanized” antibodies, chimeric antibodies, and even fully human antibodies that can be produced by phage display, gene and chromosome transfection methods, as well as by other means. This term also includes monoclonal antibodies, polyclonal antibodies, multivalent antibodies, multispecific antibodies (e.g., bispecific antibodies).

Antigen-binding specificity of an antibody can be determined by its variable regions located in the amino terminal regions of the light and heavy chains. The variable regions of a light chain and associated heavy chain form an “antigen binding domain” that recognizes a specific epitope; an antibody thus has two antigen binding domains. The antigen binding domains in a wild type antibody are directed to the same epitope of an immunogenic protein, and a single wild type antibody is thus capable of binding two molecules of the immunogenic protein at the same time. Thus, a wild type antibody is monospecific (i.e., directed to a unique antigen) and divalent (i.e., capable of binding two molecules of antigen).

“Polyclonal antibodies” can be generated in an immunogenic response to a protein having any epitopes. A “monoclonal antibody” can be developed and is a specific antibody that recognizes a single specific epitope of an immunogenic protein. A “naked antibody” is an intact antibody molecule that contains no further modifications such as conjugation with a toxin, or with a chelate for binding to a radionuclide. See, e.g., Markrides, Therapeutic inhibition of the complement system, Pharmacol. Rev. 50:59-87, 1998.

An “antibody fragment” is a portion of an intact antibody such as F(ab′)a, F(ab)2, Fab′, Fab, Fv, sFv and the like. Antibody fragments produced by limited proteolysis of wild type antibodies are called proteolytic antibody fragments.

The term “assay,” as used herein, includes any methodology useful for detecting the presence of a BRCA sequence described herein. Such methodologies include, but are not limited to, immunohistochemical and/or Western blot analysis, immunoprecipitation, molecular binding assays, ELISA. ELIFA, fluorescence activated cell sorting (FACS) and the like, quantitative blood based assays (e.g., Serum ELISA), biochemical enzymatic activity assays, in situ hybridization, Northern analysis and/or PCR analysis of mRNAs, and gent and/or tissue array analysis.

A “detection molecule” is a molecule that specifically recognizes and associates with a BRCA sequence described herein. A detection molecule is said to recognize a BRCA sequence if it specifically binds (e.g., covalently or non-covalently) to the BRCA sequence. Detection molecules include, without limitation, antibodies, aptamers, oligonucleotides (e.g., probes or primers), small molecules, and the like.

BRCA sequences include polypeptides comprising the amino acid sequences set forth in SEQ ID NOs 1-10 and any nucleic acid encoding such polypeptides.

### Production of Antibody Fragments

Some embodiments of the claimed methods and/or compositions may concern antibody fragments. Such antibody fragments may be obtained by pepsin or papain digestion of whole antibodies by conventional methods. For example, antibody fragments may be produced by enzymatic cleavage of antibodies with pepsin to provide a 5S fragment denoted F(ab′)2. This fragment may be further cleaved using a thiol reducing agent and, optionally, a blocking group for the sulfhydryl groups resulting from cleavage of disulfide linkages, to produce 3.5S Fab′ monovalent fragments. Alternatively, an enzymatic cleavage using pepsin produces two monovalent Fab fragments and an Fc fragment. Exemplary methods for producing antibody fragments are known in the art.

Other methods of cleaving antibodies, such as separation of heavy chains to form monovalent light-heavy chain fragments, hatter cleavage of fragments or other enzymatic, chemical or genetic techniques also may be used, so long as the fragments bind to the antigen that is recognized by the intact antibody. For example, Fv fragments comprise an association of V_{H} and V_{L} chains. This association can be noncovalent. Alternatively, the variable chains may be linked by an intermolecular disulfide bond or cross-linked by chemicals such as glutaraldehyde by techniques known in the art.

In another embodiment of the present invention, the humanized antibody may include a complete antibody molecule, having full length heavy and light chains; a fragment thereof, such as a Fab, Fab′, F(ab′)2, or Fv fragment; a single chain antibody fragment, e.g. a single chain Fv, a light chain or heavy chain monomer or dimer; multivalent monospecific antigen binding proteins comprising two, three, four or more antibodies or fragments thereof bound to each other by a connecting structure; or a fragment or analogue of any of these or any other molecule with the same specificity as a phosphospecific antibody, carboxy-terminal or amino-terminal binding antibody. In one particular embodiment, the antibody may include a complete antibody molecule, having full length heavy and light chains.

Any standard technique of molecular biology known in the art may be used to prepare DNA sequences coding for the antibodies according to the present invention. For example, DNA sequences may be synthesised completely or in part using oligonucleotide synthesis techniques. Site-directed mutagenesis and polymerase chain reaction (PCR) techniques may be used as appropriate. Suitable processes include the PCR strand overlap procedure and PCR mutagenesis as described in for example “PCR Technology Principles and Applications for DNA Amplification” (1989), Ed. H. A. Edith, Stockholm Press, N.Y., London, and oligonucleotide directed mutagenesis (Kramer et al, Nucleic. Acid. Res. 12 9441 (1984)).

Any expression vector known in the art can be used herein. DNA coding for human immunoglobulin sequences may be obtained by any means known in the art. The skilled artisan is aware that multiple codon sequences may encode the same amino acid and that in various embodiments, the disclosed nucleic acid sequences may be substituted with an alternative sequence that encodes the same sequence of amino acids. The skilled artisan is also aware that, depending on the species of origin for a cell line used to express a protein from a nucleic acid sequence, the codon usage may be optimized to enhance expression in the selected species. Such species preferred codon frequencies are well known in the art.

### Recombinant Fusion Proteins Containing Antibody Fragments

Nucleic acid sequences encoding antibody fragments that recognize specific epitopes can be obtained by techniques that are well known in the art. For example, hybridomas secreting antibodies of a desired specificity can be used to obtain antibody encoding DNA that can be prepared using known techniques, for example, by PCR or by traditional cDNA cloning techniques. Alternatively, Fab′ expression libraries or antibody phage display libraries can be constructed to screen for antibody fragments having a desired specificity. Any methods known in the art may be used to generate these nucleic acid sequences.

Proteins or peptides may be synthesized, in whole or in part, in solution or on a solid support in accordance with conventional techniques. Various automatic synthesizers are commercially available and can be used in accordance with known protocols. See, for example, Stewart and Young. (1984, Solid Phase Peptide Synthesis, 2d. ed., Pierce Chemical Co.); Tarn et al., (1983, J. Am, Chem. Soc., 105:6442); Merrifield, (1986, Science, 232; 341-347); and Barmy and Merrifield (1979, The Peptides, Gross and Meienhofer, eds., Academic Press, New York, pp. 1-284).

Useful diagnostic/detection agents for use in combination technologies disclosed herein include, but are not limited to, radioisotopes, dyes (such as with the biotin-streptavidin complex), radiopaque materials (e.g., iodine, barium, gallium, and thallium compounds and the like), contrast agents, fluorescent compounds or molecules and enhancing agents (e.g., paramagnetic ions) for magnetic resonance imaging (MRD. Any diagnostic/detection agent known in the art is contemplated.

Chemotherapeutic agents, for the purpose of this disclosure, can include all known chemotherapeutic agents. Some suitable chemotherapeutic agents are described in Remington's Pharmaceutical Sciences 19th Ed. (Mack Publishing Co. 1995). Other suitable chemotherapeutic agents, such as experimental drugs, are known to those of skill in the art.

In one embodiment of the present invention, a toxin may include but is not limited to ricin, abrin, ribonuclease, RNase I, Staphylococcal enterotoxin-A, pokeweed antiviral protein, gelonin, diphtherin toxin, Pseudomonas exotoxin, or Pseudomonas endotoxin.

In one embodiment of the present invention, enzymes are also useful therapeutic agents and may be selected from the group including but not limited to malate dehydrogenase, Staphylococcal nuclease, delta-V-steroid isomerase, yeast alcohol dehydrogenase, a-glycerophosphate dehydrogenase, triose phosphate isomerase, horseradish peroxidase, alkaline phosphatase, asparaginase, glucose oxidase, p-galactosidase, ribonuclease, urease, catalase, glucose-6-phosphate dehydrogenase, glucoamylase and acetylcholinesterase.

In one embodiment of the present invention, immunomodulators are also useful therapeutic agents and any known immunomodulator is contemplated.

It is contemplated herein that any of the disclosed antibodies may be used alone or in combination to detect the presence of the target protein modification associated with a tissue. In addition, it is contemplated that the antibodies disclosed herein may be used to direct a therapeutic agent to a specific tissue alone or in combination with other antibodies directed to deliver the same or a different therapeutic agent.

### Nucleic Acids

As described herein, an aspect of the present disclosure concerns isolated nucleic acids and methods of use of isolated nucleic acids, The term “nucleic acid” is intended to include DNA and RNA and can be either be double-stranded or single-stranded. In a preferred embodiment, the nucleic acid is a cDNA comprising a nucleotide sequence such as found in GenBank. In certain embodiments, the nucleic acid sequences disclosed herein have utility as hybridization probes or amplification primers. These nucleic acids may be used, for example, in diagnostic evaluation of tissue samples. In certain embodiments, these probes and primers consist of oligonucleotide fragments. Such fragments should be of sufficient length to provide specific hybridization to a RNA or DNA tissue sample. The sequences typically will be 10-20 nucleotides, but may be longer. Longer sequences greater than 50 even up to full length, are preferred for certain embodiments.

A gene or gene fragment encoding a polypeptide (e.g. BRCA2 or BRCA1 novel peptide or mutation specific protein) may be inserted into an expression vector by standard subcloning techniques. An E. coli expression vector may be used which produces the recombinant polypeptide as a fusion protein, allowing rapid affinity purification of the protein. Examples of such fusion protein expression systems are the FLAG system. (IBI, New Haven, Conn.), and the 6×His system (Qiagen, Chatsworth, Calif.).

Expression of a genetic disorder associated protein in mammalian cells may be accomplished using a mammalian expression vector. Examples of mammalian expression vectors include pCDM8 (Seed, B, (1987) Nature 329:840) and pMT2PC (Kaufman et al, (1987), EMBO J. 6:187-195). Plasmid vectors are contemplated of use herein and any methods known in the art may be used.

For applications in which the nucleic acid segments are incorporated into vectors, such as plasmids, cosmids or viruses, these segments may be combined with other DNA sequences, such as promoters, polyadenylation signals, restriction enzyme sites, multiple cloning sites, other coding segments, and the like, such that their overall length may vary considerably.

One embodiment includes isolated nucleic acids encoding proteins having biological activity of a genetic disorder-associated protein. The term “isolated” refers to a nucleic acid substantially free of cellular material or culture medium when produced by recombinant DNA techniques, or chemical precursors or other chemicals when chemically synthesized. An “isolated” nucleic acid is also tree of sequences that naturally flank the nucleic acid (i.e., sequences located at the 5′ and 3′ ends of the nucleic acid) in the organism from which the nucleic acid is derived.

It will be appreciated that isolated nucleic adds includes nucleic acids having substantial sequence homology with the nucleotide sequence of a genetic disorder-associated protein found in GenBank as disclosed in methods found herein or encoding proteins having substantial homology to the corresponding amino acid sequence (e.g. 60%, 70% or 80% homology). Proteins comprising an amino acid sequence that is 50%, 60%, 70%, 80% or 90% homologous with the novel amino acid sequences may provide proteins or peptides having a genetic disorder-associated activity or trait.

A nucleic acid of the embodiments, for instance an oligonucleotide, can also be chemically synthesized using standard techniques. Various methods of chemically synthesizing polydeoxynucleotide are known, including solid-phase synthesis which, like peptide synthesis, has been fully automated in commercially available DNA synthesizers (See i.e., Itakura et al. U.S. Pat. No. 4,598,049; Caruthers et al. U.S. Pat. No. 4,458,066; and Itakura U.S. Pat. Nos. 4,401,796 and 4,373,071).

### Protein Purification

Various methods for quantifying the degree of purification of the protein or peptide will be known to those of skill in the art in light of the present disclosure. These include, for example, determining the specific activity of an active fraction, or analysis by SDS/PAGE to identify the number of polypeptides in a given fraction. A preferred method for assessing the purity of a fraction is to calculate the specific activity of the fraction, to compare it to the specific activity of the initial extract, and to thus calculate the degree of purity, herein assessed by a “-fold purification number”. The actual units used to represent the amount of activity will be dependent upon the particular assay technique chosen to follow the purification and whether or not the expressed protein or peptide exhibits a detectable activity.

Methods for purifying various forms of proteins are known. (i.e., Protein Purification, ed. Scopes, Springer-Verlag, New York, N.Y., 1987; Methods in Molecular Biology: Protein Purification Protocols, Vol. 59, ed. Doonan, Humana Press, Totowa, N.J., 1996). Methods disclosed in the cited references are exemplary only and any variation known in the art may be used.

There is no general requirement that the protein or peptide always be provided in the most purified state. Indeed, it is contemplated that less substantially purified products will have utility in certain embodiments. Partial purification may be accomplished by using fewer purification steps in combination, or by utilizing different forms of the same general purification scheme. Methods exhibiting a lower degree of relative purification may have advantages in total recovery of protein product, or in maintaining the activity of an expressed protein.

In certain embodiments of the methods of the present invention, the subject may be a mammal such as a human or a non-human such as a wild animal, bird, reptile, a domesticated animal or farm animal.

### Kits

In still further embodiments, the present invention concerns kits for use with the methods described above. Small molecules, proteins, antibodies or peptides may be employed for use in any of the disclosed methods. In addition, other agents such as anti-bacterial agents, immunosuppressive agents, anti-inflammatory agents may be provided in the kit. The kits thus can include, in suitable container means, a protein or a peptide or analog agent, and optionally one or more additional agents. In certain embodiments, kits of the present invention can include a detection molecule of use to test a sample from a subject for levels or presence of a novel peptide or novel protein disclosed herein.

The kits may further include a suitably aliquoted composition of the encoded protein or polypeptide antigen, whether labeled or unlabeled, as may be used to prepare a standard curve for a detection assay.

The container means of the kits will generally include at least one vial, test tube, flask, bottle, syringe or other container means, into which the antibody or antigen may be placed, and preferably, suitably aliquoted. Where a second or third binding ligand or additional component is provided, the kit will also generally contain a second, third or other additional container into which this ligand or component may be placed. The kits of the present invention will also typically include a means for containing the antibody, antigen, and any other reagent containers in close confinement for commercial sale. Such containers may include injection or blow-molded plastic containers into which the desired vials are retained.

### EXAMPLES

The following examples are included to demonstrate preferred embodiments of the invention. It should be appreciated by those of skill in the art that the techniques disclosed in the examples which follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

A number of common founder mutations (Table 2) are known in BRCA1 and 2 as deletion mutants and thought to create a truncation. Analysis of these truncation mutants leads to false positive, diagnosis. Because of this it was reasoned that by generating antibodies to common founder mutations. The BRCA1 or BRCA2 mutant protein could be directly identified with an IHC assay. We surprisingly discovered that instead of truncations, these mutations were base deletions creating frameshifts. The amino acid sequence of the mutant founder proteins is strikingly different than expected. It was found that 16 novel amino acids tor BRCA1-185delAG and 21 novel amino acids for BRCA2-6174delT were discovered for example, (see Table 1). Using novel antibodies, we have identified novel peptides not previously known to be associated with the BRCA1 And 2 mutation. In one example, using a method disclosed herein, the 185delAG-BRCA1 mutant protein was identified.

TABLE 2
Common Founder Mutations In Specific Ethnic Populations

| Ethnic | | Percentage in Breast | Percentage in |
|---|---|---|---|
| population | Specific Mutation | (Ovarian) Cancer | Population |
| Ashkenazi | 185delAG BRCA1 | 50-60% (40-50%) | 1% |
| Jewish | | | |
| Hispanic | 185delAG BRCA1 | 3-15% (unknown) | Unknown |
| Ashkenazi | 6174delT BRCA2 | 30-35% (30-60%) | 2% |
| Jewish | | | |
| African | 943ins10 BRCA1 | 2-3% (unknown) | Unknown |
| American | | | |
| Polish | 5382insC, C61G | 80-92% (80-90%) | 3-4% |
| | and 4153delA | | |

Unique Sequences for Mutation Specific Antibody
Generation:
(unique amino acids shown in BOLD)

| | | |
|---|---|---|
| Wildtype BRCA1 | Sequence | KIL |
| BRCA1-185delAG | Sequence | KILVSHLSGVDQGTCLHKV |
| Wildtype BRCA2 | Sequence | GIFSTASGKSVQVS . . . |
| BRCA2-6174delT | Sequence | GIFSTARENLSRYQMLHYKT |
| | | QDKCFLK |

### Example 2

### Generation of Monoclonal Antibodies to Ashkenazi Mutations

Ashkenazi mutations BRCA1-185delAG and BRCA2-6174delT result in mRNAs which contain frame shifts. The resultant expected amino acid sequences are not present in humans in the absence of these mutations. The Ashkenazi mutations differ from the wildtype BRCA 1 and BRCA2 protein sequences, with 16 novel amino acids for BRCA1-185delAG and 21 novel amino acids for BRCA2-6174delT. Peptides containing the amino acids unique to each Ashkenazi mutation were synthesized with an added N-terminal cysteine residue, linked to keyhole limpet hemocyanin (KLH), and used to immunize mice to produce monoclonal antibodies. 15 mice were immunized for each sequence and then screened by ELISA for the mutant sequence and IHC to determine specificity for the mutant sequence in tissue. 3 mice were selected for fusions followed by generation of monoclonal antibodies. One of these antibodies was named BRCA2-11535.

### BRCA1 Mulation-Specific Antibody

Because no cell line is known to express the Ashkenazi BRCA1-185delAG, the BRCA1-185delAG-specific antibody was screened against normal non-mutant breast tissue and a breast cancer/normal breast sample from a patient with a 185delAG BRCA1 mutant hereditary breast cancer. IHC using the BRCA1-185delAG-specific antibody was performed on the breast tissues using an En Vision peroxidase IHC kit (Dako, Carpenteria, Calif.) and counterstained with hematoxylin nuclear staining using standard protocols.

The results show a marked difference in antibody staining between mutant tissues and non-mutant tissues. While the is essentially zero or minimal staining of non-mutant tissues, virtually all nuclei M both the cancer and the normal tissue of the BRCA1-185delAG mutation carrier. Some nuclei in the cancer are markedly positive and some have less positive staining, which is often seen in IHC studies and may reflect differential expression of BRCA1 message or protein in different cells. Based on this result, IHC using a BRCA1-185delAG-specific antibody distinguishes a BRCA1-185delAG mutant tissue from a non-mutant tissue.

### BRCA2 Mutation-Specific Antibody

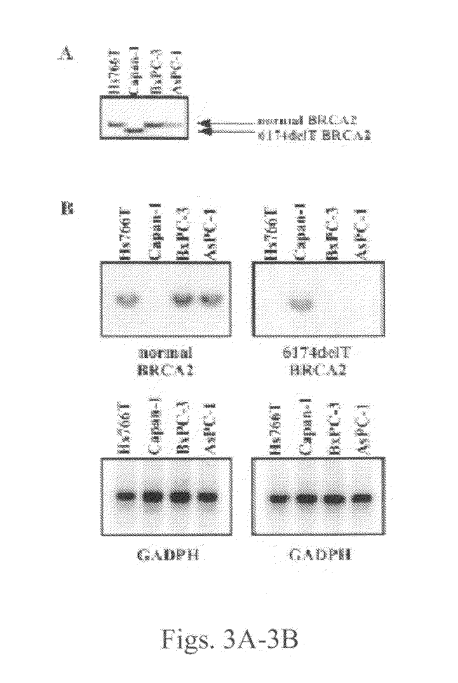

DNA samples from far pancreatic carcinoma cell lines, HS766T, Capan-1, BxPC-3 and AsPC-1 were oligoblotted using allele-specific oligonucleotides to identify cell lines that contain a BRCA2-6174delT mutation. See for example FIGS. 3A-3B. Capan-1 was identified as lacking a normal a BRCA2 and containing a BRCA2-6174delT mutation.

The BRCA2-6174delT-specific antibody, BRCA2-11535, was screened against Capan-1 cells and MCF7 control cells, which do not contain a BRCA2-6174delT mutation. IHC using BRCA2-11535 was performed on the cell lines using an EnVision peroxidase MC kit (Dako, Carpenteria, Calif.) and counterstained with hematoxylin nuclear staining using standard protocols.

The results show a marked difference in antibody staining between Capan-1 cells, which contain the Ashkenazi BRCA2-6174delT mutation, and MCF-7 cells, which do not. While there is essentially zero or minimal staining of non-mutant cells, both cytoplasm and nuclei of Capan-1 cells appear to be stained.

### Example 3

In another exemplary method, IHC using BRCA2 6174delAG mutation specific antibody was analyzed. Two panels are from Capan-1 pancreatic cancer cells which express only the 6174delT Ashkenazi BRCA2 mutation based on DNA sequencing. The other two panels are from MCF7 cells which have a non-mutant BRCA2. Brown staining represents positive antibody reaction from the Envision immunoperoxidase reagents and the blue represents the nuclear stain hematoxylin. All photomicrographs are 40× magnification.

### Testing Sensitivity and Specificity of Ashkenazi Mutation-Specific Antibodies in Paraffin Fixed Tissue Samples.

Each of the Ashkenazi mutation-specific antibodies are tested to determine their sensitivity and specificity. In order to have statistical confidence that each antibody is at least 70% sensitive, each is required to identify at least 4 of 5 patients (80%) with the appropriate Ashkenazi mutation. In order to have statistical confidence that each antibody is at least 90% specific, each is required to provide negative IHC results in 18 of 20 (90%) sporadic breast cancer samples, which are unlikely to have an Ashkenazi mutation.

Five breast cancer biopsies from patients known to have the BRCA1 Ashkenazi mutation 185delAG and 5 from patients km to have the BRCA2 Ashkenazi mutation 6174delT an obtained. In addition, 20 sporadic breast cancer samples from patients without a family history of breast cancer are obtained. The samples are provided as unstained slides, which have been stripped of identifiers and given a unique identification number so that samples cannot be identified as positive for an Ashkenazi mutation prior to IHC staining. Any sporadic breast cancer samples that produce positive IHC staining with an Ashkenazi mutation-specific antibody is tested using oligoblotting and DNA sequencing to make certain the result is not due to an unsuspected BRCA mutation.

Immunohistochemistry is performed using standard procures. The immunostaining is quantitated within the cancer and adjacent normal. For the BRCA1-185delAG-specific antibody, staining in cells will be graded from 0 to 3, where 0 is no detectable staining, 1 is a faint dot-like nuclear pattern, 2 is diffuse light staining of the entire nucleus, and 3 is dense staining of the entire nucleus. For the BRCA1-6174delT-specific antibody, staining in cells will be graded from 0 to 3, where 0 is no detectable staining, 1 is a faint dot-like staining pattern, 2 is diffuse light staining, and 3 is dense staining of the entire cell.

In addition to mutation specific antibody staining, ail tissue sections is stained with a negative control (IgG) and a positive control (Ki67). Ki67 antibody is used to verify nuclear fixation and preservation.

Scoring of the BRCA1 mutation specific antibody test is be compared to DNA sequencing results and a univariate comparison is be done using Chi-square or Fisher's exact test, as appropriate. These associations will establish diagnostic value (i.e. is able to distinguish at all between Ashkenazi mutant breast tissue and non-mutant breast tissue. Differences between screening results by mutation specific antibody test and DNA sequencing is analyzed by using the McNemar test (Gotten 2004). A P value of less than 0.05 is considered an indication of statistical significance.

### Example 4

### Using Ashkenazi Mutation-Specific Antibodies in Elba

In one exemplary method, Ashkenazi mutant BRCA proteins can be detected using ELISA. ELISA plates are produced using known methods. For example, a multi-well plate can be coated with capture antibodies. Capture antibodies can be antibodies specific for Ashkenazi mutant BRCA1 or 2 novel proteins, peptides or for wild-type BRCA proteins.

ELISA assays can be performed using known methods on a cell lysate from tissue samples of an individual at risk for developing breast cancer. Because Ashkenazi mutations are inherited, such mutations can be identified in any tissue where BRCA1 or 2 is expressed. The cell lysates are incubated with the capture antibodies in order to facilitate binding of BRCA proteins to the capture antibodies. Proteins that do not bind the capture antibodies are washed away.

A second antibody is used to detect captured BRCA1 or 2 proteins having an Ashkenazi mutation. Ifs wild-type BRCA-specific antibody is used as a capture antibody, then an Ashkenazi mutation-specific antibody is used to detect the presence of Ashkenazi mutant BRCA proteins. If an Ashkenazi mutant-specific antibody is used as the capture antibody, either an Ashkenazi mutant-specific or a wild-type-specific antibody can be used as the detection antibody. Presence of an Ashkenazi mutant BRCA protein is detected by detecting the presence of the detection antibody. The detection antibody can be labeled (e.g., with a chromophore or a fluorescent molecule) for this purpose, or a third, labeled antibody that binds the detection antibody can be used.

### Example 5

### Tissue Truncation Test for BRCA1 and BRCA2 Mutations

An IHC test to identify BRCA mutations was developed based on the idea that most BRCA1 and BRCA2 mutations are due to protein truncations. The test, termed BRCA Tissue Truncation Test, demonstrates protein truncation by using N-terminal and C-terminal antibodies to show that the beginning of the protein is present but the C-terminus is absent as shown in FIG. 2. The BRCA2 Tissue Truncation Test has 95% sensitivity and 98% specificity based on screening 24 BRCA2-6174delT mutant breast cancers and 58 sporadic breast cancers.

### Example 6

### Using Ashkenazi Mutation-Specific Antibodies to Identify Ashkenazi Mutation Carriers

Individuals with a family history of breast cancer, pancreatic cancer, ovarian cancer, and the like, or individuals diagnosed with such a cancer can be tested to determine whether BRCA is truncated. BRCA truncation can be identified in cancerous tissues using BRCA Tissue Vacation Test or other known test, such as PCR or oligoblotting tests specific for BRCA truncation.

In individuals where a cancer tissue sample is unavailable, Ashkenazi mutation-specific antibodies can be used. Unlike the BRCA Tissue Truncation Test, which demonstrates the loss of C-terminal BRCA or N-terminal BRCA peptide regions and can only be performed on cancer tissues, the Mutation Specific-Antibodies will identify the mutant protein in any tissue where BRCA is expressed in a mutant individual so buccal (cheek) cells could be screened for non-invasive testing of ethnic populations to identify mutation carriers.

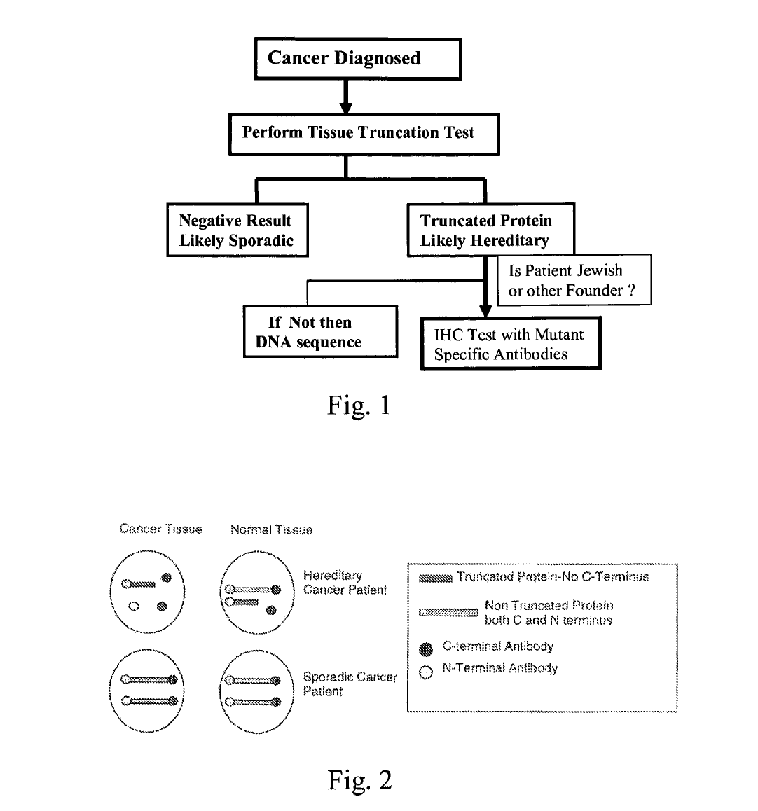

In addition, Ashkenazi mutation-specific antibodies can be used to identify what kind of BRCA mutation is present following a BRCA Tissue Truncation Test or other test. For example, if a truncation is identified using a BRCA Tissue Truncation Test, a sample can be further tested using Ashkenazi mutation-specific antibodies to determine whether the mutant BRCA contains the novel amino acids. See FIG. 1.

There are several advantages and disadvantages to using Ashkenazi mutation-specific antibodies over DNA sequencing alone. The advantages may include: 1) higher pre-test likelihood for screening protocols (since hereditary risk is 0.12%-8% in healthy individuals and is approx 3-10% in cancer patients); 2) more carriers would be identified; 3) many individuals who appear to require genetic testing could be easily excluded by testing an affected relative's cancer; and 4) cancers can be screened quickly for rational therapies at the time of diagnosis.

### Example 7

### Using Ashkenazi Mutation-specific Antibodies to Predict Treatment Efficacy

In one example, tissue samples from individuals with breast cancer, pancreatic cancer, ovarian cancer, and the like can be screened for the positive reactivity to an Ashkenazi mutation-specific antibody. In addition, the individuals can be assessed for response or lack of response to a cancer treatment. The presence or absence of positive reactivity to an Ashkenazi mutation-specific antibody can be correlated to cancer treatment response or lack of response. An individual's cancer can be predicted to respond or not respond to a particular career treatment based on the identified correlations. Such predictions can be used to identify treatments with the greatest potential efficacy for an individual with cancer.

### Example 8

### Using Ashkenazi Mutation-Specific Antibodies to Purify and Characterize Ashkenazi Mutant Proteins

In one exemplary method. Ashkenazi mutation-specific antibodies can be used to purify Ashkenazi mutant proteins using known methods. For example, Ashkenazi mutant proteins can be isolated using immunoprecipitation, affinity chromatography, and the like.

Immunoprecipitation is performed by linking Ashkenazi mutation-specific antibodies to agarose beads, magnetic beads, or the like, and incubating the linked antibodies with a cell lysate containing, the appropriate Ashkenazi mutant protein. Proteins are immunoprecipitated from antibody-lysate mixture using known methods, such as centrifugation or magnetic bead capture using a magnet. Unbound proteins are washed from the beads using one or more washes and the beads are again precipitated. Immunoprecipitated proteins are resolved on a polyacrylamide gel, purified from the gel, and sequenced using mass spectrometry, Edman degradation, or the like. Cell lysates containing Ashkenazi mutant BRCA proteins are obtained from cancer tissue samples or cell lines, such as Capan-1, or from recombinant cells expressing an Ashkenazi mutant nucleic acid.

Affinity chromatography is performed by coupling Ashkenazi mutation-specific antibodies to a gel matrix in a chromatography column. Cell lysates containing the appropriate Ashkenazi mutant protein are passed through the column to allow binding of the mutant BRCA proteins to the matrix-bound antibodies. Unbound proteins are washed from the column. The Ashkenazi mutant protein is eluted from the column using known techniques such as washing the column with a denaturing wash, and the protein is collected and sequenced.

BRCA1 sequence alignments, unique sequences underlined

| | | | |
|---|---|---|---|
| SEQ ID NO: 11 | 1 | MDLSALRVEEVQNVINAMQKILECPI-------CLELIKEPVSTKCDHIFCKFCMLKLLNQKKGPSQCPLCKNDITKRSL | 73 |
| SEQ ID NO: 9 | 1 | MDLSALRVEEVQNVINAMQKILECPI-------CLELIKEPVSTKCDHIFCKFCMLKLLNQKKGPSQCPLCKNDITKRSL | 73 |
| SEQ ID NO: 10 | 1 | MDLSALRVEEVQNVINAMQKILECPI-------CLELIKEPVSTKCDHIFCKFCMLKLLNQKKGPSQCPLCKNDITKRSL | 73 |
| SEQ ID NO: 8 | 1 | MDLSALRVEEVQNVINAMQKILECPI-------CLELIKEPVSTKCDHIFCKFCMLKLLNQKKGPSQCPLCKNDITKRSL | 73 |
| SEQ ID NO: 6 | 1 | MDLSALRVEEVQNVINAMQKILVSHLSGVDQGTCLHKV------------------------------------------ | 38 |
| SEQ ID NO: 11 | 74 | QESTRFSQLVEELLKIICAFQLDTGLEYANSYNFAKKENNSPEHLKDEVSIIQSMGYRNRAKRLLQSEPENPSLQETSLS | 153 |
| SEQ ID NO: 9 | 74 | QESTRFSQLVEELLKIICAFQLDTGLEYANSYNFAKKENNSPEHLKDEVSIIQSMGYRNRAKRLLQSEPENPSLQETSLS | 153 |
| SEQ ID NO: 10 | 74 | QESTRFSQLVEELLKIICAFQLDTGLEYANSYNFAKKENNSPEHLKDEVSIIQSMGYRNRAKRLLQSEPENPSLQETSLS | 153 |
| SEQ ID NO: 8 | 74 | QESTRFSQLVEELLKIICAFQLDTGLEYANSYNFAKKENNSPEHLKDEVSIIQSMGYRNRAKRLLQSEPENPSLQETSLS | 153 |
| SEQ ID NO: 6 | | -------------------------------------------------------------------------------- | |
| SEQ ID NO: 11 | 154 | VQLSNLGTVRTLRTKQRIQPQKTSVYIELGSDSSEDTVNKATYCSVGDQELLQITPQGTRDEISLDSAKKAACEFSETDV | 233 |
| SEQ ID NO: 9 | 154 | VQLSNLGTVRTLRTKQRIQPQKTSVYIELGSDSSEDTVNKATYCSVGDQELLQITPQGTRDEISLDSAKKAACEFSETDV | 233 |
| SEQ ID NO: 10 | 154 | VQLSNLGTVRTLRTKQRIQPQKTSVYIELGSDSSEDTVNKATYCSVGDQELLQITPQGTRDEISLDSAKKAACEFSETDV | 233 |
| SEQ ID NO: 8 | 154 | VQLSNLGTVRTLRTKQRIQPQKTSVYIELGSDSSEDTVNKATYCSVGDQELLQITPQGTRDEISLDSAKKAACEFSETDV | 233 |
| SEQ ID NO: 6 | | -------------------------------------------------------------------------------- | |
| SEQ ID NO: 11 | 234 | TNTEHHQPSNNDLNTTEKRAAERHPEKYQGSSVSNLHVEPCGTNTHASSLQHENSSLLLTKDRMNVEKAEFCNKSKQPGL | 313 |
| SEQ ID NO: 9 | 234 | TNTEHHQPSNNDLNTTEKRAAERHPEKYQGSSVSNLHVEPCGTNTHASSLQHENSSLLLTKDRMNVEKAEFCNKSKQPGL | 313 |
| SEQ ID NO: 10 | 234 | TNTEHHQPSNNDLNTTEKRAAERHPEKYQGSSVSNLHVEPCGTNTHASSLQHENSSLLLTKDRMNVEKAEFCNKSKQPGL | 313 |
| SEQ ID NO: 8 | 234 | TNTEHHQPSNNDLNTTEKRAAERHPEKYQGSSVSNLHVEPCGAMWHKYSCQLITA------------------------- | 288 |
| SEQ ID NO: 6 | | -------------------------------------------------------------------------------- | |
| SEQ ID NO: 11 | 314 | ARSQHNRWAGSKETCNDRRTPSTEKKVDLNADPLCERKEWNKQKLPCSENPRDTEDVPWITLNSSIQKVNEWFSRSDELL | 393 |
| SEQ ID NO: 9 | 314 | ARSQHNRWAGSKETCNDRRTPSTEKKVDLNADPLCERKEWNKQKLPCSENPRDTEDVPWITLNSSIQKVNEWFSRSDELL | 393 |
| SEQ ID NO: 10 | 314 | ARSQHNRWAGSKETCNDRRTPSTEKKVDLNADPLCERKEWNKQKLPCSENPRDTEDVPWITLNSSIQKVNEWFSRSDELL | 393 |
| SEQ ID NO: 8 | | -------------------------------------------------------------------------------- | |
| SEQ ID NO: 6 | | -------------------------------------------------------------------------------- | |
| SEQ ID NO: 11 | 394 | GSDDSHDGESESNAKVADVLDVLNEVDEYSGSSEKIDLLASDPHEALICKSERVHSKSVESNIEDKIFGKTYRKKASLPN | 473 |
| SEQ ID NO: 9 | 394 | GSDDSHDGESESNAKVADVLDVLNEVDEYSGSSEKIDLLASDPHEALICKSERVHSKSVESNIEDKIFGKTYRKKASLPN | 473 |
| SEQ ID NO: 10 | 394 | GSDDSHDGESESNAKVADVLDVLNEVDEYSGSSEKIDLLASDPHEALICKSERVHSKSVESNIEDKIFGKTYRKKASLPN | 473 |
| SEQ ID NO: 8 | | -------------------------------------------------------------------------------- | |
| SEQ ID NO: 6 | | -------------------------------------------------------------------------------- | |
| SEQ ID NO: 11 | 474 | LSHVTENLIIGAFVTEPQIIQERPLTNKLKRKRRPTSGLHPEDFIKKADLAVQKTPEMINQGTNQTEQNGQVMNITNSGH | 553 |
| SEQ ID NO: 9 | 474 | LSHVTENLIIGAFVTEPQIIQERPLTNKLKRKRRPTSGLHPEDFIKKADLAVQKTPEMINQGTNQTEQNGQVMNITNSGH | 553 |
| SEQ ID NO: 10 | 474 | LSHVTENLIIGAFVTEPQIIQERPLTNKLKRKRRPTSGLHPEDFIKKADLAVQKTPEMINQGTNQTEQNGQVMNITNSGH | 553 |
| SEQ ID NO: 8 | | -------------------------------------------------------------------------------- | |
| SEQ ID NO: 6 | | -------------------------------------------------------------------------------- | |
| SEQ ID NO: 11 | 554 | ENKTKGDSIQNEKNPNPIESLEKESAFKTKAEPISSSISNMELELNIHNSKAPKKNRLRRKSSTRHIHALELVVSRNLSP | 633 |
| SEQ ID NO: 9 | 554 | ENKTKGDSIQNEKNPNPIESLEKESAFKTKAEPISSSISNMELELNIHNSKAPKKNRLRRKSSTRHIHALELVVSRNLSP | 633 |
| SEQ ID NO: 10 | 554 | ENKTKGDSIQNEKNPNPIESLEKESAFKTKAEPISSSISNMELELNIHNSKAPKKNRLRRKSSTRHIHALELVVSRNLSP | 633 |
| SEQ ID NO: 8 | | -------------------------------------------------------------------------------- | |
| SEQ ID NO: 6 | | -------------------------------------------------------------------------------- | |
| SEQ ID NO: 11 | 634 | PNCTELQIDSCSSSEEIKKKKYNQMPVRHSRNLQLMEGKEPATGAKKSNKPNEQTSKRHDSDTFPELKLTNAPGSFTKCS | 713 |
| SEQ ID NO: 9 | 634 | PNCTELQIDSCSSSEEIKKKKYNQMPVRHSRNLQLMEGKEPATGAKKSNKPNEQTSKRHDSDTFPELKLTNAPGSFTKCS | 713 |
| SEQ ID NO: 10 | 634 | PNCTELQIDSCSSSEEIKKKKYNQMPVRHSRNLQLMEGKEPATGAKKSNKPNEQTSKRHDSDTFPELKLTNAPGSFTKCS | 713 |
| SEQ ID NO: 8 | | -------------------------------------------------------------------------------- | |
| SEQ ID NO: 6 | | -------------------------------------------------------------------------------- | |
| SEQ ID NO: 11 | 714 | NTSELKEFVNPSLPREEKEEKLETVKVSNNAEDPKDLMLSGERVLQTERSVESSSISLVPGTDYGTQESISLLEVSTLGK | 793 |
| SEQ ID NO: 9 | 714 | NTSELKEFVNPSLPREEKEEKLETVKVSNNAEDPKDLMLSGERVLQTERSVESSSISLVPGTDYGTQESISLLEVSTLGK | 793 |
| SEQ ID NO: 10 | 714 | NTSELKEFVNPSLPREEKEEKLETVKVSNNAEDPKDLMLSGERVLQTERSVESSSISLVPGTDYGTQESISLLEVSTLGK | 793 |
| SEQ ID NO: 8 | | -------------------------------------------------------------------------------- | |
| SEQ ID NO: 6 | | -------------------------------------------------------------------------------- | |
| SEQ ID NO: 11 | 794 | AKTEPNKCVSQCAAFENPKGLIHGCSKDNRNDTEGFKYPLGHEVNHSRETSIEMEESELDAQYLQNTFKVSKRQSFAPFS | 873 |
| SEQ ID NO: 9 | 794 | AKTEPNKCVSQCAAFENPKGLIHGCSKDNRNDTEGFKYPLGHEVNHSRETSIEMEESELDAQYLQNTFKVSKRQSFAPFS | 873 |
| SEQ ID NO: 10 | 794 | AKTEPNKCVSQCAAFENPKGLIHGCSKDNRNDTEGFKYPLGHEVNHSRETSIEMEESELDAQYLQNTFKVSKRQSFAPFS | 873 |
| SEQ ID NO: 8 | | -------------------------------------------------------------------------------- | |
| SEQ ID NO: 6 | | -------------------------------------------------------------------------------- | |
| SEQ ID NO: 11 | 874 | NPGNAEEECATFSAHSGSLKKQSPKVTFECEQKEENQGKNESNIKPVQTVNITAGFPVVGQKDKPVDNAKCSIKGGSRFC | 953 |
| SEQ ID NO: 9 | 874 | NPGNAEEECATFSAHSGSLKKQSPKVTFECEQKEENQGKNESNIKPVQTVNITAGFPVVGQKDKPVDNAKCSIKGGSRFC | 953 |
| SEQ ID NO: 10 | 874 | NPGNAEEECATFSAHSGSLKKQSPKVTFECEQKEENQGKNESNIKPVQTVNITAGFPVVGQKDKPVDNAKCSIKGGSRFC | 953 |
| SEQ ID NO: 8 | | -------------------------------------------------------------------------------- | |
| SEQ ID NO: 6 | | -------------------------------------------------------------------------------- | |
| SEQ ID NO: 11 | 954 | LSSQFRGNETGLITPNKHGLLQNPYRIPPLFPIKSFVKTKCKKNLLEENFEEHSMSPEREMGNENIPSTVSTISRNNIRE | 1033 |
| SEQ ID NO: 9 | 954 | LSSQFRGNETGLITPNKHGLLQNPYRIPPLFPIKSFVKTKCKKNLLEENFEEHSMSPEREMGNENIPSTVSTISRNNIRE | 1033 |
| SEQ ID NO: 10 | 954 | LSSQFRGNETGLITPNKHGLLQNPYRIPPLFPIKSFVKTKCKKNLLEENFEEHSMSPEREMGNENIPSTVSTISRNNIRE | 1033 |
| SEQ ID NO: 8 | | -------------------------------------------------------------------------------- | |
| SEQ ID NO: 6 | | -------------------------------------------------------------------------------- | |
| SEQ ID NO: 11 | 1034 | NVFKEASSSNINEVGSSTNEVGSSINEIGSSDENIQAELGRNRGPKLNAMLRLGVLQPEVYKQSLPGSNCKHPEIKKQEY | 1113 |
| SEQ ID NO: 9 | 1034 | NVFKEASSSNINEVGSSTNEVGSSINEIGSSDENIQAELGRNRGPKLNAMLRLGVLQPEVYKQSLPGSNCKHPEIKKQEY | 1113 |
| SEQ ID NO: 10 | 1034 | NVFKEASSSNINEVGSSTNEVGSSINEIGSSDENIQAELGRNRGPKLNAMLRLGVLQPEVYKQSLPGSNCKHPEIKKQEY | 1113 |
| SEQ ID NO: 8 | | -------------------------------------------------------------------------------- | |
| SEQ ID NO: 6 | | -------------------------------------------------------------------------------- | |
| SEQ ID NO: 11 | 1114 | EEVVQTVNTDFSPYLISDNLEQPMGSSHASQVCSETPDDLLDDGEIKEDTSFAENDIKESSAVFSKSVQKGELSRSPSPF | 1193 |
| SEQ ID NO: 9 | 1114 | EEVVQTVNTDFSPYLISDNLEQPMGSSHASQVCSETPDDLLDDGEIKEDTSFAENDIKESSAVFSKSVQKGELSRSPSPF | 1193 |
| SEQ ID NO: 10 | 1114 | EEVVQTVNTDFSPYLISDNLEQPMGSSHASQVCSETPDDLLDDGEIKEDTSFAENDIKESSAVFSKSVQKGELSRSPSPF | 1193 |
| SEQ ID NO: 8 | | -------------------------------------------------------------------------------- | |
| SEQ ID NO: 6 | | -------------------------------------------------------------------------------- | |
| SEQ ID NO: 11 | 1194 | THTHLAQGYRRGAKKLESSEENLSSEDEELPCFQHLLFGKVNNIPSQSTRHSTVATECLSKNTEENLLSLKNSLNDCSNQ | 1273 |
| SEQ ID NO: 9 | 1194 | THTHLAQGYRRGAKKLESSEENLSSEDEELPCFQHLLFGKVNNIPSQSTRHSTVATECLSKNTEENLLSLKNSLNDCSNQ | 1273 |
| SEQ ID NO: 10 | 1194 | THTHLAQGYRRGAKKLESSEENLSSEDEELPCFQHLLFGKVNNIPSQSTRHSTVATECLSKNTEENLLSLKNSLNDCSNQ | 1273 |
| SEQ ID NO: 8 | | -------------------------------------------------------------------------------- | |
| SEQ ID NO: 6 | | -------------------------------------------------------------------------------- | |
| SEQ ID NO: 11 | 1274 | VILAKASQEHHLSEETKCSASLFSSQCSELEDLTANTNTQDPFLIGSSKQMRHQSESQGVGLSDKELVSDDEERGTGLEE | 1353 |
| SEQ ID NO: 9 | 1274 | VILAKASQEHHLSEETKCSASLFSSQCSELEDLTANTNTQDPFLIGSSKQMRHQSESQGVGLSDKELVSDDEERGTGLEE | 1353 |
| SEQ ID NO: 10 | 1274 | VILAKASQEHHLSEETKCSASLFSSQCSELEDLTANTNTQDPFLIGSSKQMRHQSESQGVGLSDKELVSDDEKEERAWKK | 1353 |
| SEQ ID NO: 8 | | -------------------------------------------------------------------------------- | |
| SEQ ID NO: 6 | | -------------------------------------------------------------------------------- | |
| SEQ ID NO: 11 | 1354 | NNQEEQSMDSNLGEAASGCESETSVSEDCSGLSSQSDILTTQQRDTMQHNLIKLQQEMAELEAVLEQHGSQPSNSYPSII | 1433 |
| SEQ ID NO: 9 | 1354 | NNQEEQSMDSNLGEAASGCESETSVSEDCSGLSSQSDILTTQQRDTMQHNLIKLQQEMAELEAVLEQHGSQPSNSYPSII | 1433 |
| SEQ ID NO: 10 | 1354 | IIKKSKAWIQT--------------------------------------------------------------------- | 1364 |
| SEQ ID NO: 8 | | -------------------------------------------------------------------------------- | |
| SEQ ID NO: 6 | | -------------------------------------------------------------------------------- | |
| SEQ ID NO: 11 | 1434 | SDSSALEDLRNPEQSTSEKAVLTSQKSSEYPISQNPEGLSADKFEVSADSSTSKNKEPGVERSSPSKCPSLDDRWYMHSC | 1513 |
| SEQ ID NO: 9 | 1434 | SDSSALEDLRNPEQSTSEKAVLTSQKSSEYPISQNPEGLSADKFEVSADSSTSKNKEPGVERSSPSKCPSLDDRWYMHSC | 1513 |
| SEQ ID NO: 10 | | -------------------------------------------------------------------------------- | |
| SEQ ID NO: 8 | | -------------------------------------------------------------------------------- | |
| SEQ ID NO: 6 | | -------------------------------------------------------------------------------- | |
| SEQ ID NO: 11 | 1514 | SGSLQNRNYPSQEELIKVVDVEEQQLEESGPHDLTETSYLPRQDLEGTPYLESGISLFSDDPESDPSEDRAPESARVGNI | 1593 |
| SEQ ID NO: 9 | 1514 | SGSLQNRNYPSQEELIKVVDVEEQQLEESGPHDLTETSYLPRQDLEGTPYLESGISLFSDDPESDPSEDRAPESARVGNI | 1593 |
| SEQ ID NO: 10 | | -------------------------------------------------------------------------------- | |
| SEQ ID NO: 8 | | -------------------------------------------------------------------------------- | |
| SEQ ID NO: 6 | | -------------------------------------------------------------------------------- | |
| SEQ ID NO: 11 | 1594 | PSSTSALKVPQLKVAESAQSPAAAHTTDTAGYNAMEESVSREKPELTASTERVNKRMSMVVSGLTPEEFMLVYKFARKHH | 1673 |
| SEQ ID NO: 9 | 1594 | PSSTSALKVPQLKVAESAQSPAAAHTTDTAGYNAMEESVSREKPELTASTERVNKRMSMVVSGLTPEEFMLVYKFARKHH | 1673 |
| SEQ ID NO: 10 | | -------------------------------------------------------------------------------- | |
| SEQ ID NO: 8 | | -------------------------------------------------------------------------------- | |
| SEQ ID NO: 6 | | -------------------------------------------------------------------------------- | |
| SEQ ID NO: 11 | 1674 | ITLTNLITEETTHVVMKTDAEFVCERTLKYFLGIAGGKWVVSYFWVTQSIKERKMLNEHDFEVRGDVVNGRNHQGPKRAR | 1753 |
| SEQ ID NO: 9 | 1674 | ITLTNLITEETTHVVMKTDAEFVCERTLKYFLGIAGGKWVVSYFWVTQSIKERKMLNEHDFEVRGDVVNGRNHQGPKRAR | 1753 |
| SEQ ID NO: 10 | | -------------------------------------------------------------------------------- | |
| SEQ ID NO: 8 | | -------------------------------------------------------------------------------- | |
| SEQ ID NO: 6 | | -------------------------------------------------------------------------------- | |
| SEQ ID NO: 11 | 1754 | ESQDRKIFRGLEICCYGPFTNMPTDQLEWMVQLCGASVVKELSSFTLGTGVHPIVVVQPDAWTEDNGFHAIGQMCEAPVV | 1833 |
| SEQ ID NO: 9 | 1754 | ESPGQKDLQGARNLLLWALHQHAHRSTGMDGTAVWCFCGEGAFIIHPWHRCPPNCGCAARCLDRGQWLPCNWADV----- | 1828 |
| SEQ ID NO: 10 | | -------------------------------------------------------------------------------- | |
| SEQ ID NO: 8 | | -------------------------------------------------------------------------------- | |
| SEQ ID NO: 6 | | -------------------------------------------------------------------------------- | |
| SEQ ID NO: 11 | 1834 | TREWVLDSVALYQCQELDTYLIPQIPHSHY | 1863 |
| SEQ ID NO: 9 | | ------------------------------ | |
| SEQ ID NO: 10 | | ------------------------------ | |
| SEQ ID NO: 8 | | ------------------------------ | |
| SEQ ID NO: 6 | | ------------------------------ | |

BRCA2 sequences, unique sequence underlined

| | | | |
|---|---|---|---|
| SEQ ID NO: 12 | 1 | MPIGSKERPTFFEIFKTRCNKADLGPISLNWFEELSSEAPPYNSEPAEESEHKNNNYEPNLFKTPQRKPSYNQLASTPII | 80 |
| SEQ ID NO: 7 | 1 | MPIGSKERPTFFEIFKTRCNKADLGPISLNWFEELSSEAPPYNSEPAEESEHKNNNYEPNLFKTPQRKPSYNQLASTPII | 80 |
| SEQ ID NO: 12 | 81 | FKEQGLTLPLYQSPVKELDKFKLDLGRNVPNSRHKSLRTVKTKMDQADDVSCPLLNSCLSESPVVLQCTHVTPQRDKSVV | 160 |
| SEQ ID NO: 7 | 81 | FKEQGLTLPLYQSPVKELDKFKLDLGRNVPNSRHKSLRTVKTKMDQADDVSCPLLNSCLSESPVVLQCTHVTPQRDKSVV | 160 |
| SEQ ID NO: 12 | 161 | CGSLFHTPKFVKGRQTPKHISESLGAEVDPDMSWSSSLATPPTLSSTVLIVRNEEASETVFPHDTTANVKSYFSNHDESL | 240 |
| SEQ ID NO: 7 | 161 | CGSLFHTPKFVKGRQTPKHISESLGAEVDPDMSWSSSLATPPTLSSTVLIVRNEEASETVFPHDTTANVKSYFSNHDESL | 240 |
| SEQ ID NO: 12 | 241 | KKNDRFIASVTDSENTNQREAASHGFGKTSGNSFKVNSCKDHIGKSMPNVLEDEVYETVVDTSEEDSFSLCFSKCRTKNL | 320 |
| SEQ ID NO: 7 | 241 | KKNDRFIASVTDSENTNQREAASHGFGKTSGNSFKVNSCKDHIGKSMPNVLEDEVYETVVDTSEEDSFSLCFSKCRTKNL | 320 |
| SEQ ID NO: 12 | 321 | QKVRTSKTRKKIFHEANADECEKSKNQVKEKYSFVSEVEPNDTDPLDSNVANQKPFESGSDKISKEVVPSLACEWSQLTL | 400 |
| SEQ ID NO: 7 | 321 | QKVRTSKTRKKIFHEANADECEKSKNQVKEKYSFVSEVEPNDTDPLDSNVANQKPFESGSDKISKEVVPSLACEWSQLTL | 400 |
| SEQ ID NO: 12 | 401 | SGLNGAQMEKIPLLHISSCDQNISEKDLLDTENKRKKDFLTSENSLPRISSLPKSEKPLNEETVVNKRDEEQHLESHTDC | 480 |
| SEQ ID NO: 7 | 401 | SGLNGAQMEKIPLLHISSCDQNISEKDLLDTENKRKKDFLTSENSLPRISSLPKSEKPLNEETVVNKRDEEQHLESHTDC | 480 |
| SEQ ID NO: 12 | 481 | ILAVKQAISGTSPVASSFQGIKKSIFRIRESPKETFNASFSGHMTDPNFKKETEASESGLEIHTVCSQKEDSLCPNLIDN | 560 |
| SEQ ID NO: 7 | 481 | ILAVKQAISGTSPVASSFQGIKKSIFRIRESPKETFNASFSGHMTDPNFKKETEASESGLEIHTVCSQKEDSLCPNLIDN | 560 |
| SEQ ID NO: 12 | 561 | GSWPATTTQNSVALKNAGLISTLKKKTNKFIYAIHDETSYKGKKIPKDQKSELINCSAQFEANAFEAPLTFANADSGLLH | 640 |
| SEQ ID NO: 7 | 561 | GSWPATTTQNSVALKNAGLISTLKKKTNKFIYAIHDETSYKGKKIPKDQKSELINCSAQFEANAFEAPLTFANADSGLLH | 640 |
| SEQ ID NO: 12 | 641 | SSVKRSCSQNDSEEPTLSLTSSFGTILRKCSRNETCSNNTVISQDLDYKEAKCNKEKLQLFITPEADSLSCLQEGQCEND | 720 |
| SEQ ID NO: 7 | 641 | SSVKRSCSQNDSEEPTLSLTSSFGTILRKCSRNETCSNNTVISQDLDYKEAKCNKEKLQLFITPEADSLSCLQEGQCEND | 720 |
| SEQ ID NO: 12 | 721 | PKSKKVSDIKEEVLAAACHPVQHSKVEYSDTDFQSQKSLLYDHENASTLILTPTSKDVLSNLVMISRGKESYKMSDKLKG | 800 |
| SEQ ID NO: 7 | 721 | PKSKKVSDIKEEVLAAACHPVQHSKVEYSDTDFQSQKSLLYDHENASTLILTPTSKDVLSNLVMISRGKESYKMSDKLKG | 800 |
| SEQ ID NO: 12 | 801 | NNYESDVELTKNIPMEKNQDVCALNENYKNVELLPPEKYMRVASPSRKVQFNQNTNLRVIQKNQEETTSISKITVNPDSE | 880 |
| SEQ ID NO: 7 | 801 | NNYESDVELTKNIPMEKNQDVCALNENYKNVELLPPEKYMRVASPSRKVQFNQNTNLRVIQKNQEETTSISKITVNPDSE | 880 |
| SEQ ID NO: 12 | 881 | ELFSDNENNFVFQVANERNNLALGNTKELHETDLTCVNEPIFKNSTMVLYGDTGDKQATQVSIKKDLVYVLAEENKNSVK | 960 |
| SEQ ID NO: 7 | 881 | ELFSDNENNFVFQVANERNNLALGNTKELHETDLTCVNEPIFKNSTMVLYGDTGDKQATQVSIKKDLVYVLAEENKNSVK | 960 |
| SEQ ID NO: 12 | 961 | QHIKMTLGQDLKSDISLNIDKIPEKNNDYMNKWAGLLGPISNHSFGGSFRTASNKEIKLSEHNIKKSKMFFKDIEEQYPT | 1040 |
| SEQ ID NO: 7 | 961 | QHIKMTLGQDLKSDISLNIDKIPEKNNDYMNKWAGLLGPISNHSFGGSFRTASNKEIKLSEHNIKKSKMFFKDIEEQYPT | 1040 |
| SEQ ID NO: 12 | 1041 | SLACVEIVNTLALDNQKKLSKPQSINTVSAHLQSSVVVSDCKNSHITPQMLFSKQDFNSNHNLTPSQKAEITELSTILEE | 1120 |
| SEQ ID NO: 7 | 1041 | SLACVEIVNTLALDNQKKLSKPQSINTVSAHLQSSVVVSDCKNSHITPQMLFSKQDFNSNHNLTPSQKAEITELSTILEE | 1120 |
| SEQ ID NO: 12 | 1121 | SGSQFEFTQFRKPSYILQKSTFEVPENQMTILKTTSEECRDADLHVIMNAPSIGQVDSSKQFEGTVEIKRKFAGLLKNDC | 1200 |
| SEQ ID NO: 7 | 1121 | SGSQFEFTQFRKPSYILQKSTFEVPENQMTILKTTSEECRDADLHVIMNAPSIGQVDSSKQFEGTVEIKRKFAGLLKNDC | 1200 |
| SEQ ID NO: 12 | 1201 | NKSASGYLTDENEVGFRGFYSAHGTKLNVSTEALQKAVKLFSDIENISEETSAEVHPISLSSSKCHDSVVSMFKIENHND | 1280 |
| SEQ ID NO: 7 | 1201 | NKSASGYLTDENEVGFRGFYSAHGTKLNVSTEALQKAVKLFSDIENISEETSAEVHPISLSSSKCHDSVVSMFKIENHND | 1280 |
| SEQ ID NO: 12 | 1281 | KTVSEKNNKCQLILQNNIEMTTGTFVEEITENYKRNTENEDNKYTAASRNSHNLEFDGSDSSKNDTVCIHKDETDLLFTD | 1360 |
| SEQ ID NO: 7 | 1281 | KTVSEKNNKCQLILQNNIEMTTGTFVEEITENYKRNTENEDNKYTAASRNSHNLEFDGSDSSKNDTVCIHKDETDLLFTD | 1360 |
| SEQ ID NO: 12 | 1361 | QHNICLKLSGQFMKEGNTQIKEDLSDLTFLEVAKAQEACHGNTSNKEQLTATKTEQNIKDFETSDTFPQTASGKNISVAK | 1440 |
| SEQ ID NO: 7 | 1361 | QHNICLKLSGQFMKEGNTQIKEDLSDLTFLEVAKAQEACHGNTSNKEQLTATKTEQNIKDFETSDTFPQTASGKNISVAK | 1440 |
| SEQ ID NO: 12 | 1441 | ESFNKIVNFFDQKPEELHNFSLNSELHSDIRKNKMDILSYEETDIVKHKILKESVPVGTGNQLVTFQGQPERDEKIKEPT | 1520 |
| SEQ ID NO: 7 | 1441 | ESFNKIVNFFDQKPEELHNFSLNSELHSDIRKNKMDILSYEETDIVKHKILKESVPVGTGNQLVTFQGQPERDEKIKEPT | 1520 |
| SEQ ID NO: 12 | 1521 | LLGFHTASGKKVKIAKESLDKVKNLFDEKEQGTSEITSFSHQWAKTLKYREACKDLELACETIEITAAPKCKEMQNSLNN | 1600 |
| SEQ ID NO: 7 | 1521 | LLGFHTASGKKVKIAKESLDKVKNLFDEKEQGTSEITSFSHQWAKTLKYREACKDLELACETIEITAAPKCKEMQNSLNN | 1600 |
| SEQ ID NO: 12 | 1601 | DKNLVSIETVVPPKLLSDNLCRQTENLKTSKSIFLKVKVHENVEKETAKSPATCYTNQSPYSVIENSALAFYTSCSRKTS | 1680 |
| SEQ ID NO: 7 | 1601 | DKNLVSIETVVPPKLLSDNLCRQTENLKTSKSIFLKVKVHENVEKETAKSPATCYTNQSPYSVIENSALAFYTSCSRKTS | 1680 |
| SEQ ID NO: 12 | 1681 | VSQTSLLEAKKWLREGIFDGQPERINTADYVGNYLYENNSNSTIAENDKNHLSEKQDTYLSNSSMSNSYSYHSDEVYNDS | 1760 |
| SEQ ID NO: 7 | 1681 | VSQTSLLEAKKWLREGIFDGQPERINTADYVGNYLYENNSNSTIAENDKNHLSEKQDTYLSNSSMSNSYSYHSDEVYNDS | 1760 |
| SEQ ID NO: 12 | 1761 | GYLSKNKLDSGIEPVLKNVEDQKNTSFSKVISNVKDANAYPQTVNEDICVEELVTSSSPCKNKNAAIKLSISNSNNFEVG | 1840 |
| SEQ ID NO: 7 | 1761 | GYLSKNKLDSGIEPVLKNVEDQKNTSFSKVISNVKDANAYPQTVNEDICVEELVTSSSPCKNKNAAIKLSISNSNNFEVG | 1840 |
| SEQ ID NO: 12 | 1841 | PPAFRIASGKIVCVSHETIKKVKDIFTDSFSKVIKENNENKSKICQTKIMAGCYEALDDSEDILHNSLDNDECSTHSHKV | 1920 |
| SEQ ID NO: 7 | 1841 | PPAFRIASGKIVCVSHETIKKVKDIFTDSFSKVIKENNENKSKICQTKIMAGCYEALDDSEDILHNSLDNDECSTHSHKV | 1920 |
| SEQ ID NO: 12 | 1921 | FADIQSEEILQHNQNMSGLEKVSKISPCDVSLETSDICKCSIGKLHKSVSSANTCGIFSTASGKSVQVSDASLQNARQVF | 2000 |
| SEQ ID NO: 7 | 1921 | FADIQSEEILQHNQNMSGLEKVSKISPCDVSLETSDICKCSIGKLHKSVSSANTCGIFSTARENLSRYQMLHYKTQDKCF | 2000 |
| SEQ ID NO: 12 | 2001 | SEIEDSTKQVFSKVLFKSNEHSDQLTREENTAIRTPEHLISQKGFSYNVVNSSAFSGFSTASGKQVSILESSLHKVKGVL | 2080 |
| SEQ ID NO: 7 | 2001 | LK------------------------------------------------------------------------------ | 2002 |
| SEQ ID NO: 12 | 2081 | EEFDLIRTEHSLHYSPTSRQNVSKILPRVDKRNPEHCVNSEMEKTCSKEFKLSNNLNVEGGSSENNHSIKVSPYLSQFQQ | 2160 |
| SEQ ID NO: 7 | | -------------------------------------------------------------------------------- | |
| SEQ ID NO: 12 | 2161 | DKQQLVLGTKVSLVENIHVLGKEQASPKNVKMEIGKTETFSDVPVKTNIEVCSTYSKDSENYFETEAVEIAKAFMEDDEL | 2240 |
| SEQ ID NO: 7 | | -------------------------------------------------------------------------------- | |
| SEQ ID NO: 12 | 2241 | TDSKLPSHATHSLFTCPENEEMVLSNSRIGKRRGEPLILVGEPSIKRNLLNEFDRIIENQEKSLKASKSTPDGTIKDRRL | 2320 |
| SEQ ID NO: 7 | | -------------------------------------------------------------------------------- | |
| SEQ ID NO: 12 | 2321 | FMHHVSLEPITCVPFRTTKERQEIQNPNFTAPGQEFLSKSHLYEHLTLEKSSSNLAVSGHPFYQVSATRNEKMRHLITTG | 2400 |
| SEQ ID NO: 7 | | -------------------------------------------------------------------------------- | |
| SEQ ID NO: 12 | 2401 | RPTKVFVPPFKTKSHFHRVEQCVRNINLEENRQKQNIDGHGSDDSKNKINDNEIHQFNKNNSNQAAAVTFTKCEEEPLDL | 2480 |
| SEQ ID NO: 7 | | -------------------------------------------------------------------------------- | |
| SEQ ID NO: 12 | 2481 | ITSLQNARDIQDMRIKKKQRQRVFPQPGSLYLAKTSTLPRISLKAAVGGQVPSACSHKQLYTYGVSKHCIKINSKNAESF | 2560 |
| SEQ ID NO: 7 | | -------------------------------------------------------------------------------- | |
| SEQ ID NO: 12 | 2561 | QFHTEDYFGKESLWTGKGIQLADGGWLIPSNDGKAGKEEFYRALCDTPGVDPKLISRIWVYNHYRWIIWKLAAMECAFPK | 2640 |
| SEQ ID NO: 7 | | -------------------------------------------------------------------------------- | |
| SEQ ID NO: 12 | 2641 | EFANRCLSPERVLLQLKYRYDTEIDRSRRSAIKKIMERDDTAAKTLVLCVSDIISLSANISETSSNKTSSADTQKVAIIE | 2720 |
| SEQ ID NO: 7 | | -------------------------------------------------------------------------------- | |
| SEQ ID NO: 12 | 2721 | LTDGWYAVKAQLDPPLLAVLKNGRLTVGQKIILHGAELVGSPDACTPLEAPESLMLKISANSTRPARWYTKLGFFPDPRP | 2800 |
| SEQ ID NO: 7 | | -------------------------------------------------------------------------------- | |
| SEQ ID NO: 12 | 2801 | FPLPLSSLFSDGGNVGCVDVIIQRAYPIQWMEKTSSGLYIFRNEREEEKEAAKYVEAQQKRLEALFTKIQEEFEEHEENT | 2880 |
| SEQ ID NO: 7 | | -------------------------------------------------------------------------------- | |
| SEQ ID NO: 12 | 2881 | TKPYLPSRALTRQQVRALQDGAELYEAVKNAADPAYLEGYFSEEQLRALNNHRQMLNDKKQAQIQLEIRKAMESAEQKEQ | 2960 |
| SEQ ID NO: 7 | | -------------------------------------------------------------------------------- | |
| SEQ ID NO: 12 | 2961 | GLSRDVTTVWKLRIVSYSKKEKDSVILSIWRPSSDLYSLLTEGKRYRIYHLATSKSKSKSERANIQLAATKKTQYQQLPV | 3040 |
| SEQ ID NO: 7 | | -------------------------------------------------------------------------------- | |
| SEQ ID NO: 12 | 3041 | SDEILFQIYQPREPLHFSKFLDPDFQPSCSEVDLIGFVVSVVKKTGLAPFVYLSDECYNLLAIKFWIDLNEDIIKPHMLI | 3120 |
| SEQ ID NO: 7 | | -------------------------------------------------------------------------------- | |
| SEQ ID NO: 12 | 3121 | AASNLQWRPESKSGLLTLFAGDFSVFSASPKEGHFQETFNKMKNTVENIDILCNEAENKLMHILHANDPKWSTPTKDCTS | 3200 |
| SEQ ID NO: 7 | | -------------------------------------------------------------------------------- | |
| SEQ ID NO: 12 | 3201 | GPYTAQIIPGTGNKLLMSSPNCEIYYQSPLSLCMAKRKSVSTPVSAQMTSKSCKGEKEIDDQKNCKKRRALDFLSRLPLP | 3280 |
| SEQ ID NO: 7 | | -------------------------------------------------------------------------------- | |
| SEQ ID NO: 12 | 3281 | PPVSPICTFVSPAAQKAFQPPRSCGTKYETPIKKKELNSPQMTPFKKENEISLLESNSIADEELALINTQALLSGSTGEK | 3360 |
| SEQ ID NO: 7 | | -------------------------------------------------------------------------------- | |
| SEQ ID NO: 12 | 3361 | QFISVSESTRTAPTSSEDYLRLKRRCTTSLIKEQESSQASTEECEKNKQDTITTKKYI | 3418 |
| SEQ ID NO: 7 | | ---------------------------------------------------------- | |

All of the COMPOSITIONS and METHODS disclosed and claimed herein may be made and executed without undue experimentation in light of the present disclosure. While the COMPOSITIONS and METHODS have been described in terms of preferred embodiments, it will be apparent to those of skill in the art that variation may be applied to the COMPOSITIONS and METHODS and in the steps or in the sequence of steps of the METHODS described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

What is claimed:

1. An isolated polypeptide comprising amino acid sequences of SEQ ID NO: 1, 2, 3, 4 or 5.

2. The isolated polypeptide of claim 1, wherein the amino acid sequence is one of amino acid sequences of SEQ ID NO: 1, 2, 3, 4 or 5.

3. An isolated polypeptide comprising the amino acid sequence of SEQ ID NO: 6, 7, 8, 9 or 10.

4. An isolated polynucleotide encoding the polypeptide of claim 1, or a complement thereof.

5. A vector comprising the polynucleotide of claim 4.

6. A transgenic cell comprising the polynucleotide of claim 4.

7. A method of making the isolated polynucleotide of claim 4, comprising amplifying genomic DNA isolated from a sample obtained from a subject.

8. A detection molecule that recognizes and associates with the polypeptide of claim 1 or the polynucleotide of claim 4.

9. The detection molecule of claim 8, wherein the molecule is an antibody or antigen-binding fragment thereof, a sense or antisense oligonucleotide, or an aptamer.

10. An antibody or fragment thereof that specifically recognizes the polypeptide of any of claims 1.

11. The antibody of claim 10, wherein said antibody is a monoclonal, polyclonal, chimeric, humanized, or conjugated antibody.

12. An assay system comprising the detection molecule of claim 8.

13. The assay system of claim 12, wherein said detection molecule comprises the antibody or fragment of claim 11.

14. The assay system of claim 12, wherein said detection molecule comprises a sense or antisense oligonucleotide probe or primer.

15. A method for diagnosis of breast cancer in a patient, comprising:
analyzing a sample obtained from the patient for the presence or absence of the polypeptide of claim 1 or the polynucleotide of claim 4;
wherein the presence of the polypeptide or polynucleotide in the sample indicates breast cancer in the patient.

16. A method for identifying a patient at risk of developing breast cancer, comprising:
analyzing a sample obtained from the patient for the presence or absence of the polypeptide of claim 1 or the polynucleotide of claim 4;
wherein presence of the polypeptide or polynucleotide in the sample increases the risk of developing breast cancer in the patient compared to a control sample absent the polypeptide or polynucleotide.

17. A method for evaluating prognosis of a breast cancer patient, comprising:
analyzing a sample obtained from the patient for an amount of the polypeptide of claim 1 or the polynucleotide of claim 4 and comparing the amount with a control sample;
wherein a decrease in the amount of the polypeptide or polynucleotide in the sample versus control is indicative of improvement in the patient's disease and an increase in the amount is indicative of progression of the patient's disease.

18. A kit comprising:
a) a detection molecule that recognizes and associates with the polypeptide of claim 1 or the polynucleotide of claim 4; and
b) a suitable container.

19. The kit of claim 18, wherein the detection molecule is an antibody or antigen-binding fragment thereof, a sense or antisense oligonucleotide, or aptamer.

20. A method comprising:
a) obtaining haploid cells from a subject not previously diagnosed with a disorder;
b) obtaining one or more N-terminal antibodies to a first target protein of a control sample wherein haploid cells and the control sample are of the same origin;
c) obtaining one or more carboxy-terminal (C-terminal) antibodies to a second target protein of a control sample wherein the haploid cells and the control sample are of the same origin;
d) exposing the haploid cells to the one or more N-terminal and C-terminal antibodies and allowing the antibodies to bind to the haploid cells;
e) detecting the one or more antibodies bound to the haploid cells; and
f) assessing presence or risk of developing the disorder based on the level of bound antibodies to the target proteins of the tissue sample(s) compared to bound antibodies to the target proteins of a control sample.

21. The method of claim 20, where identifying the risk of developing the disorder is used for genetic counseling or to select haploid cells which lack an abnormality for the purpose for producing offspring free of the specific genetic disease.

22. The method of claim 20, where the antibodies are used to enrich for sperm or oocytes which do not contain the truncated or mutant protein.


SEQUENCE LISTING (12 sequences)

SEQ ID NO: 1 - protein, 16 aa, Homo sapiens
  PEPTIDE (1)..(16): BRCA1 novel peptide
VSHLSGVDQGTCLHKV

SEQ ID NO: 2 - protein, 21 aa, Homo sapiens
  PEPTIDE (1)..(21): BRCA2 novel peptide
RENLSRYQMLHYKTQDKCFLK

SEQ ID NO: 3 - protein, 13 aa, Homo sapiens
  PEPTIDE (1)..(13): BRCA1 novel peptide
AMWHKYSCQLITA

SEQ ID NO: 4 - protein, 73 aa, Homo sapiens
  PEPTIDE (1)..(73): BRCA1 novel peptide
PGQKDLQGARNLLLWALHQHAHRSTGMDGTAVWCFCGEGAFIIHPWHRCPPNCGCAARCLDRGQWLPCNWADV

SEQ ID NO: 5 - protein, 19 aa, Homo sapiens
  PEPTIDE (1)..(19): BRCA1 novel peptide
KEERAWKKIIKKSKAWIQT

SEQ ID NO: 6 - protein, 38 aa, Homo sapiens
  PEPTIDE (1)..(38): BRCA1 related peptide
MDLSALRVEEVQNVINAMQKILVSHLSGVDQGTCLHKV

SEQ ID NO: 7 - protein, 2002 aa, Homo sapiens
  PEPTIDE (1)..(2002): BRCA2 related peptide
MPIGSKERPTFFEIFKTRCNKADLGPISLNWFEELSSEAPPYNSEPAEESEHKNNNYEPNLFKTPQRKPSYNQLASTPIIFKEQGLTLPLYQSPVKELDKFKLDLGRNVPNSRHKSLRTVKTKMDQADDVSCPLLNSCLSESPVVLQCTHVTPQRDKSVVCGSLFHTPKFVKGRQTPKHISESLGAEVDPDMSWSSSLATPPTLSSTVLIVRNEEASETVFPHDTTANVKSYFSNHDESLKKNDRFIASVTDSENTNQREAASHGFGKTSGNSFKVNSCKDHIGKSMPNVLEDEVYETVVDTSEEDSFSLCFSKCRTKNLQKVRTSKTRKKIFHEANADECEKSKNQVKEKYSFVSEVEPNDTDPLDSNVANQKPFESGSDKISKEVVPSLACEWSQLTLSGLNGAQMEKIPLLHISSCDQNISEKDLLDTENKRKKDFLTSENSLPRISSLPKSEKPLNEETVVNKRDEEQHLESHTDCILAVKQAISGTSPVASSFQGIKKSIFRIRESPKETFNASFSGHMTDPNFKKETEASESGLEIHTVCSQKEDSLCPNLIDNGSWPATTTQNSVALKNAGLISTLKKKTNKFIYAIHDETSYKGKKIPKDQKSELINCSAQFEANAFEAPLTFANADSGLLHSSVKRSCSQNDSEEPTLSLTSSFGTILRKCSRNETCSNNTVISQDLDYKEAKCNKEKLQLFITPEADSLSCLQEGQCENDPKSKKVSDIKEEVLAAACHPVQHSKVEYSDTDFQSQKSLLYDHENASTLILTPTSKDVLSNLVMISRGKESYKMSDKLKGNNYESDVELTKNIPMEKNQDVCALNENYKNVELLPPEKYMRVASPSRKVQFNQNTNLRVIQKNQEETTSISKITVNPDSEELFSDNENNFVFQVANERNNLALGNTKELHETDLTCVNEPIFKNSTMVLYGDTGDKQATQVSIKKDLVYVLAEENKNSVKQHIKMTLGQDLKSDISLNIDKIPEKNNDYMNKWAGLLGPISNHSFGGSFRTASNKEIKLSEHNIKKSKMFFKDIEEQYPTSLACVEIVNTLALDNQKKLSKPQSINTVSAHLQSSVVVSDCKNSHITPQMLFSKQDFNSNHNLTPSQKAEITELSTILEESGSQFEFTQFRKPSYILQKSTFEVPENQMTILKTTSEECRDADLHVIMNAPSIGQVDSSKQFEGTVEIKRKFAGLLKNDCNKSASGYLTDENEVGFRGFYSAHGTKLNVSTEALQKAVKLFSDIENISEETSAEVHPISLSSSKCHDSVVSMFKIENHNDKTVSEKNNKCQLILQNNIEMTTGTFVEEITENYKRNTENEDNKYTAASRNSHNLEFDGSDSSKNDTVCIHKDETDLLFTDQHNICLKLSGQFMKEGNTQIKEDLSDLTFLEVAKAQEACHGNTSNKEQLTATKTEQNIKDFETSDTFFQTASGKNISVAKESFNKIVNFFDQKPEELHNFSLNSELHSDIRKNKMDILSYEETDIVKHKILKESVPVGTGNQLVTFQGQPERDEKIKEPTLLGFHTASGKKVKIAKESLDKVKNLFDEKEQGTSEITSFSHQWAKTLKYREACKDLELACETIEITAAPKCKEMQNSLNNDKNLVSIETVVPPKLLSDNLCRQTENLKTSKSIFLKVKVHENVEKETAKSPATCYTNQSPYSVIENSALAFYTSCSRKTSVSQTSLLEAKKWLREGIFDGQPERINTADYVGNYLYENNSNSTIAENDKNHLSEKQDTYLSNSSMSNSYSYHSDEVYNDSGYLSKNKLDSGIEPVLKNVEDQKNTSFSKVISNVKDANAYPQTVNEDICVEELVTSSSPCKNKNAAIKLSISNSNNFEVGPPAFRIASGKIVCVSHETIKKVKDIFTDSFSKVIKENNENKSKICQTKIMAGCYEALDDSEDILHNSLDNDECSTHSHKVFADIQSEEILQHNQNMSGLEKVSKISPCDVSLETSDICKCSIGKLHKSVSSANTCGIFSTARENLSRYQMLHYKTQDKCFLK

SEQ ID NO: 8 - protein, 288 aa, Homo sapiens
  PEPTIDE (1)..(288): BRCA1 related peptide
MDLSALRVEEVQNVINAMQKILECPICLELIKEPVSTKCDHIFCKFCMLKLLNQKKGPSQCPLCKNDITKRSLQESTRFSQLVEELLKIICAFQLDTGLEYANSYNFAKKENNSPEHLKDEVSIIQSMGYRNRAKRLLQSEPENPSLQETSLSVQLSNLGTVRTLRTKQRIQPQKTSVYIELGSDSSEDTVNKATYCSVGDQELLQITPQGTRDEISLDSAKKAACEFSETDVTNTEHHQPSNNDLNTTEKRAAERHPEKYQGSSVSNLHVEPCGAMWHKYSCQLITA

SEQ ID NO: 9 - protein, 1828 aa, Homo sapiens
  PEPTIDE (1)..(1828): BRCA1 related peptide
MDLSALRVEEVQNVINAMQKILECPICLELIKEPVSTKCDHIFCKFCMLKLLNQKKGPSQCPLCKNDITKRSLQESTRFSQLVEELLKIICAFQLDTGLEYANSYNFAKKENNSPEHLKDEVSIIQSMGYRNRAKRLLQSEPENPSLQETSLSVQLSNLGTVRTLRTKQRIQPQKTSVYIELGSDSSEDTVNKATYCSVGDQELLQITPQGTRDEISLDSAKKAACEFSETDVTNTEHHQPSNNDLNTTEKRAAERHPEKYQGSSVSNLHVEPCGTNTHASSLQHENSSLLLTKDRMNVEKAEFCNKSKQPGLARSQHNRWAGSKETCNDRRTPSTEKKVDLNADPLCERKEWNKQKLPCSENPRDTEDVPWITLNSSIQKVNEWFSRSDELLGSDDSHDGESESNAKVADVLDVLNEVDEYSGSSEKIDLLASDPHEALICKSERVHSKSVESNIEDKIFGKTYRKKASLPNLSHVTENLIIGAFVTEPQIIQERPLTNKLKRKRRPTSGLHPEDFIKKADLAVQKTPEMINQGTNQTEQNGQVMNITNSGHENKTKGDSIQNEKNPNPIESLEKESAFKTKAEPISSSISNMELELNIHNSKAPKKNRLRRKSSTRHIHALELVVSRNLSPPNCTELQIDSCSSSEEIKKKKYNQMPVRHSRNLQLMEGKEPATGAKKSNKPNEQTSKRHDSDTFPELKLTNAPGSFTKCSNTSELKEFVNPSLPREEKEEKLETVKVSNNAEDPKDLMLSGERVLQTERSVESSSISLVPGTDYGTQESISLLEVSTLGKAKTEPNKCVSQCAAFENPKGLIHGCSKDNRNDTEGFKYPLGHEVNHSRETSIEMEESELDAQYLQNTFKVSKRQSFAPFSNPGNAEEECATFSAHSGSLKKQSPKVTFECEQKEENQGKNESNIKPVQTVNITAGFPVVGQKDKPVDNAKCSIKGGSRFCLSSQFRGNETGLITPNKHGLLQNPYRIPPLFPIKSFVKTKCKKNLLEENFEEHSMSPEREMGNENIPSTVSTISRNNIRENVFKEASSSNINEVGSSTNEVGSSINEIGSSDENIQAELGRNRGPKLNAMLRLGVLQPEVYKQSLPGSNCKHPEIKKQEYEEVVQTVNTDFSPYLISDNLEQPMGSSHASQVCSETPDDLLDDGEIKEDTSFAENDIKESSAVFSKSVQKGELSRSPSPFTHTHLAQGYRRGAKKLESSEENLSSEDEELPCFQHLLFGKVNNIPSQSTRHSTVATECLSKNTEENLLSLKNSLNDCSNQVILAKASQEHHLSEETKCSASLFSSQCSELEDLTANTNTQDPFLIGSSKQMRHQSESQGVGLSDKELVSDDEERGTGLEENNQEEQSMDSNLGEAASGCESETSVSEDCSGLSSQSDILTTQQRDTMQHNLIKLQQEMAELEAVLEQHGSQPSNSYPSIISDSSALEDLRNPEQSTSEKAVLTSQKSSEYPISQNPEGLSADKFEVSADSSTSKNKEPGVERSSPSKCPSLDDRWYMHSCSGSLQNRNYPSQEELIKVVDVEEQQLEESGPHDLTETSYLPRQDLEGTPYLESGISLFSDDPESDPSEDRAPESARVGNIPSSTSALKVPQLKVAESAQSPAAAHTTDTAGYNAMEESVSREKPELTASTERVNKRMSMVVSGLTPEEFMLVYKFARKHHITLTNLITEETTHVVMKTDAEFVCERTLKYFLGIAGGKWVVSYFWVTQSIKERKMLNEHDFEVRGDVVNGRNHQGPKRARESPGQKDLQGARNLLLWALHQHAHRSTGMDGTAVWCFCGEGAFIIHPWHRCPPNCGCAARCLDRGQWLPCNWADV

SEQ ID NO: 10 - protein, 1364 aa, Homo sapiens
  PEPTIDE (1)..(1364): BRCA1 related peptide
MDLSALRVEEVQNVINAMQKILECPICLELIKEPVSTKCDHIFCKFCMLKLLNQKKGPSQCPLCKNDITKRSLQESTRFSQLVEELLKIICAFQLDTGLEYANSYNFAKKENNSPEHLKDEVSIIQSMGYRNRAKRLLQSEPENPSLQETSLSVQLSNLGTVRTLRTKQRIQPQKTSVYIELGSDSSEDTVNKATYCSVGDQELLQITPQGTRDEISLDSAKKAACEFSETDVTNTEHHQPSNNDLNTTEKRAAERHPEKYQGSSVSNLHVEPCGTNTHASSLQHENSSLLLTKDRMNVEKAEFCNKSKQPGLARSQHNRWAGSKETCNDRRTPSTEKKVDLNADPLCERKEWNKQKLPCSENPRDTEDVPWITLNSSIQKVNEWFSRSDELLGSDDSHDGESESNAKVADVLDVLNEVDEYSGSSEKIDLLASDPHEALICKSERVHSKSVESNIEDKIFGKTYRKKASLPNLSHVTENLIIGAFVTEPQIIQERPLTNKLKRKRRPTSGLHPEDFIKKADLAVQKTPEMINQGTNQTEQNGQVMNITNSGHENKTKGDSIQNEKNPNPIESLEKESAFKTKAEPISSSISNMELELNIHNSKAPKKNRLRRKSSTRHIHALELVVSRNLSPPNCTELQIDSCSSSEEIKKKKYNQMPVRHSRNLQLMEGKEPATGAKKSNKPNEQTSKRHDSDTFPELKLTNAPGSFTKCSNTSELKEFVNPSLPREEKEEKLETVKVSNNAEDPKDLMLSGERVLQTERSVESSSISLVPGTDYGTQESISLLEVSTLGKAKTEPNKCVSQCAAFENPKGLIHGCSKDNRNDTEGFKYPLGHEVNHSRETSIEMEESELDAQYLQNTFKVSKRQSFAPFSNPGNAEEECATFSAHSGSLKKQSPKVTFECEQKEENQGKNESNIKPVQTVNITAGFPVVGQKDKPVDNAKCSIKGGSRFCLSSQFRGNETGLITPNKHGLLQNPYRIPPLFPIKSFVKTKCKKNLLEENFEEHSMSPEREMGNENIPSTVSTISRNNIRENVFKEASSSNINEVGSSTNEVGSSINEIGSSDENIQAELGRNRGPKLNAMLRLGVLQPEVYKQSLPGSNCKHPEIKKQEYEEVVQTVNTDFSPYLISDNLEQPMGSSHASQVCSETPDDLLDDGEIKEDTSFAENDIKESSAVFSKSVQKGELSRSPSPFTHTHLAQGYRRGAKKLESSEENLSSEDEELPCFQHLLFGKVNNIPSQSTRHSTVATECLSKNTEENLLSLKNSLNDCSNQVILAKASQEHHLSEETKCSASLFSSQCSELEDLTANTNTQDPFLIGSSKQMRHQSESQGVGLSDKELVSDDEKEERAWKKIIKKSKAWIQT

SEQ ID NO: 11 - protein, 1863 aa, Homo sapiens
  PEPTIDE (1)..(1863): Human BRCA1
MDLSALRVEEVQNVINAMQKILECPICLELIKEPVSTKCDHIFCKFCMLKLLNQKKGPSQCPLCKNDITKRSLQESTRFSQLVEELLKIICAFQLDTGLEYANSYNFAKKENNSPEHLKDEVSIIQSMGYRNRAKRLLQSEPENPSLQETSLSVQLSNLGTVRTLRTKQRIQPQKTSVYIELGSDSSEDTVNKATYCSVGDQELLQITPQGTRDEISLDSAKKAACEFSETDVTNTEHHQPSNNDLNTTEKRAAERHPEKYQGSSVSNLHVEPCGTNTHASSLQHENSSLLLTKDRMNVEKAEFCNKSKQPGLARSQHNRWAGSKETCNDRRTPSTEKKVDLNADPLCERKEWNKQKLPCSENPRDTEDVPWITLNSSIQKVNEWFSRSDELLGSDDSHDGESESNAKVADVLDVLNEVDEYSGSSEKIDLLASDPHEALICKSERVHSKSVESNIEDKIFGKTYRKKASLPNLSHVTENLIIGAFVTEPQIIQERPLTNKLKRKRRPTSGLHPEDFIKKADLAVQKTPEMINQGTNQTEQNGQVMNITNSGHENKTKGDSIQNEKNPNPIESLEKESAFKTKAEPISSSISNMELELNIHNSKAPKKNRLRRKSSTRHIHALELVVSRNLSPPNCTELQIDSCSSSEEIKKKKYNQMPVRHSRNLQLMEGKEPATGAKKSNKPNEQTSKRHDSDTFPELKLTNAPGSFTKCSNTSELKEFVNPSLPREEKEEKLETVKVSNNAEDPKDLMLSGERVLQTERSVESSSISLVPGTDYGTQESISLLEVSTLGKAKTEPNKCVSQCAAFENPKGLIHGCSKDNRNDTEGFKYPLGHEVNHSRETSIEMEESELDAQYLQNTFKVSKRQSFAPFSNPGNAEEECATFSAHSGSLKKQSPKVTFECEQKEENQGKNESNIKPVQTVNITAGFPVVGQKDKPVDNAKCSIKGGSRFCLSSQFRGNETGLITPNKHGLLQNPYRIPPLFPIKSFVKTKCKKNLLEENFEEHSMSPEREMGNENIPSTVSTISRNNIRENVFKEASSSNINEVGSSTNEVGSSINEIGSSDENIQAELGRNRGPKLNAMLRLGVLQPEVYKQSLPGSNCKHPEIKKQEYEEVVQTVNTDFSPYLISDNLEQPMGSSHASQVCSETPDDLLDDGEIKEDTSFAENDIKESSAVFSKSVQKGELSRSPSPFTHTHLAQGYRRGAKKLESSEENLSSEDEELPCFQHLLFGKVNNIPSQSTRHSTVATECLSKNTEENLLSLKNSLNDCSNQVILAKASQEHHLSEETKCSASLFSSQCSELEDLTANTNTQDPFLIGSSKQMRHQSESQGVGLSDKELVSDDEERGTGLEENNQEEQSMDSNLGEAASGCESETSVSEDCSGLSSQSDILTTQQRDTMQHNLIKLQQEMAELEAVLEQHGSQPSNSYPSIISDSSALEDLRNPEQSTSEKAVLTSQKSSEYPISQNPEGLSADKFEVSADSSTSKNKEPGVERSSPSKCPSLDDRWYMHSCSGSLQNRNYPSQEELIKVVDVEEQQLEESGPHDLTETSYLPRQDLEGTPYLESGISLFSDDPESDPSEDRAPESARVGNIPSSTSALKVPQLKVAESAQSPAAAHTTDTAGYNAMEESVSREKPELTASTERVNKRMSMVVSGLTPEEFMLVYKFARKHHITLTNLITEETTHVVMKTDAEFVCERTLKYFLGIAGGKWVVSYFWVTQSIKERKMLNEHDFEVRGDVVNGRNHQGPKRARESQDRKIFRGLEICCYGPFTNMPTDQLEWMVQLCGASVVKELSSFTLGTGVHPIVVVQPDAWTEDNGFHAIGQMCEAPVVTREWVLDSVALYQCQELDTYLIPQIPHSHY

SEQ ID NO: 12 - protein, 3418 aa, Homo sapiens
  PEPTIDE (1)..(3418): Human BRCA2
MPIGSKERPTFFEIFKTRCNKADLGPISLNWFEELSSEAPPYNSEPAEESEHKNNNYEPNLFKTPQRKPSYNQLASTPIIFKEQGLTLPLYQSPVKELDKFKLDLGRNVPNSRHKSLRTVKTKMDQADDVSCPLLNSCLSESPVVLQCTHVTPQRDKSVVCGSLFHTPKFVKGRQTPKHISESLGAEVDPDMSWSSSLATPPTLSSTVLIVRNEEASETVFPHDTTANVKSYFSNHDESLKKNDRFIASVTDSENTNQREAASHGFGKTSGNSFKVNSCKDHIGKSMPNVLEDEVYETVVDTSEEDSFSLCFSKCRTKNLQKVRTSKTRKKIFHEANADECEKSKNQVKEKYSFVSEVEPNDTDPLDSNVANQKPFESGSDKISKEVVPSLACEWSQLTLSGLNGAQMEKIPLLHISSCDQNISEKDLLDTENKRKKDFLTSENSLPRISSLPKSEKPLNEETVVNKRDEEQHLESHTDCILAVKQAISGTSPVASSFQGIKKSIFRIRESPKETFNASFSGHMTDPNFKKETEASESGLEIHTVCSQKEDSLCPNLIDNGSWPATTTQNSVALKNAGLISTLKKKTNKFIYAIHDETSYKGKKIPKDQKSELINCSAQFEANAFEAPLTFANADSGLLHSSVKRSCSQNDSEEPTLSLTSSFGTILRKCSRNETCSNNTVISQDLDYKEAKCNKEKLQLFITPEADSLSCLQEGQCENDPKSKKVSDIKEEVLAAACHPVQHSKVEYSDTDFQSQKSLLYDHENASTLILTPTSKDVLSNLVMISRGKESYKMSDKLKGNNYESDVELTKNIPMEKNQDVCALNENYKNVELLPPEKYMRVASPSRKVQFNQNTNLRVIQKNQEETTSISKITVNPDSEELFSDNENNFVFQVANERNNLALGNTKELHETDLTCVNEPIFKNSTMVLYGDTGDKQATQVSIKKDLVYVLAEENKNSVKQHIKMTLGQDLKSDISLNIDKIPEKNNDYMNKWAGLLGPISNHSFGGSFRTASNKEIKLSEHNIKKSKMFFKDIEEQYPTSLACVEIVNTLALDNQKKLSKPQSINTVSAHLQSSVVVSDCKNSHITPQMLFSKQDFNSNHNLTPSQKAEITELSTILEESGSQFEFTQFRKPSYILQKSTFEVPENQMTILKTTSEECRDADLHVIMNAPSIGQVDSSKQFEGTVEIKRKFAGLLKNDCNKSASGYLTDENEVGFRGFYSAHGTKLNVSTEALQKAVKLFSDIENISEETSAEVHPISLSSSKCHDSVVSMFKIENHNDKTVSEKNNKCQLILQNNIEMTTGTFVEEITENYKRNTENEDNKYTAASRNSHNLEFDGSDSSKNDTVCIHKDETDLLFTDQHNICLKLSGQFMKEGNTQIKEDLSDLTFLEVAKAQEACHGNTSNKEQLTATKTEQNIKDFETSDTFFQTASGKNISVAKESFNKIVNFFDQKPEELHNFSLNSELHSDIRKNKMDILSYEETDIVKHKILKESVPVGTGNQLVTFQGQPERDEKIKEPTLLGFHTASGKKVKIAKESLDKVKNLFDEKEQGTSEITSFSHQWAKTLKYREACKDLELACETIEITAAPKCKEMQNSLNNDKNLVSIETVVPPKLLSDNLCRQTENLKTSKSIFLKVKVHENVEKETAKSPATCYTNQSPYSVIENSALAFYTSCSRKTSVSQTSLLEAKKWLREGIFDGQPERINTADYVGNYLYENNSNSTIAENDKNHLSEKQDTYLSNSSMSNSYSYHSDEVYNDSGYLSKNKLDSGIEPVLKNVEDQKNTSFSKVISNVKDANAYPQTVNEDICVEELVTSSSPCKNKNAAIKLSISNSNNFEVGPPAFRIASGKIVCVSHETIKKVKDIFTDSFSKVIKENNENKSKICQTKIMAGCYEALDDSEDILHNSLDNDECSTHSHKVFADIQSEEILQHNQNMSGLEKVSKISPCDVSLETSDICKCSIGKLHKSVSSANTCGIFSTASGKSVQVSDASLQNARQVFSEIEDSTKQVFSKVLFKSNEHSDQLTREENTAIRTPEHLISQKGFSYNVVNSSAFSGFSTASGKQVSILESSLHKVKGVLEEFDLIRTEHSLHYSPTSRQNVSKILPRVDKRNPEHCVNSEMEKTCSKEFKLSNNLNVEGGSSENNHSIKVSPYLSQFQQDKQQLVLGTKVSLVENIHVLGKEQASPKNVKMEIGKTETFSDVPVKTNIEVCSTYSKDSENYFETEAVEIAKAFMEDDELTDSKLPSHATHSLFTCPENEEMVLSNSRIGKRRGEPLILVGEPSIKRNLLNEFDRIIENQEKSLKASKSTPDGTIKDRRLFMHHVSLEPITCVPFRTTKERQEIQNPNFTAPGQEFLSKSHLYEHLTLEKSSSNLAVSGHPFYQVSATRNEKMRHLITTGRPTKVFVPPFKTKSHFHRVEQCVRNINLEENRQKQNIDGHGSDDSKNKINDNEIHQFNKNNSNQAAAVTFTKCEEEPLDLITSLQNARDIQDMRIKKKQRQRVFPQPGSLYLAKTSTLPRISLKAAVGGQVPSACSHKQLYTYGVSKHCIKINSKNAESFQFHTEDYFGKESLWTGKGIQLADGGWLIPSNDGKAGKEEFYRALCDTPGVDPKLISRIWVYNHYRWIIWKLAAMECAFPKEFANRCLSPERVLLQLKYRYDTEIDRSRRSAIKKIMERDDTAAKTLVLCVSDIISLSANISETSSNKTSSADTQKVAIIELTDGWYAVKAQLDPPLLAVLKNGRLTVGQKIILHGAELVGSPDACTPLEAPESLMLKISANSTRPARWYTKLGFFPDPRPFPLPLSSLFSDGGNVGCVDVIIQRAYPIQWMEKTSSGLYIFRNEREEEKEAAKYVEAQQKRLEALFTKIQEEFEEHEENTTKPYLPSRALTRQQVRALQDGAELYEAVKNAADPAYLEGYFSEEQLRALNNHRQMLNDKKQAQIQLEIRKAMESAEQKEQGLSRDVTTVWKLRIVSYSKKEKDSVILSIWRPSSDLYSLLTEGKRYRIYHLATSKSKSKSERANIQLAATKKTQYQQLPVSDEILFQIYQPREPLHFSKFLDPDFQPSCSEVDLIGFVVSVVKKTGLAPFVYLSDECYNLLAIKFWIDLNEDIIKPHMLIAASNLQWRPESKSGLLTLFAGDFSVFSASPKEGHFQETFNKMKNTVENIDILCNEAENKLMHILHANDPKWSTPTKDCTSGPYTAQIIPGTGNKLLMSSPNCEIYYQSPLSLCMAKRKSVSTPVSAQMTSKSCKGEKEIDDQKNCKKRRALDFLSRLPLPPPVSPICTFVSPAAQKAFQPPRSCGTKYETPIKKKELNSPQMTPFKKFNEISLLESNSIADEELALINTQALLSGSTGEKQFISVSESTRTAPTSSEDYLRLKRRCTTSLIKEQESSQASTEECEKNKQDTITTKKYI